(19) United States
(12) Patent Application Publication
(10) Pub. No.: US 2023/0265133 A1
(43) Pub. Date: Aug. 24, 2023

(54) HIGHLY POROUS SPIDER SILK FIBERS

(71) Applicant: SEEVIX MATERIAL SCIENCES LTD., Jerusalem (IL)

(72) Inventors: Haim Meir GILAD, Sde Boaz (IL); Gregory IDELSON, Maale Adumim (IL); Konstantin PRESS, Rishon Le-Zion (IL); Alon MEIR, Jerusalem (IL); Noa HADAR, Givat Brenner (IL)

(73) Assignee: SEEVIX MATERIAL SCIENCES LTD., Jerusalem (IL)

(21) Appl. No.: 18/014,547

(22) PCT Filed: Jul. 5, 2021
(86) PCT No.: PCT/IL2021/050827

§ 371 Date: Jan. 5, 2023

Related U.S. Application Data
(60) Provisional application No. 63/134,343, filed on Jan. 6, 2021.

(30) Foreign Application Priority Data
Jul. 5, 2020 (WO) PCT/IL2020/050752

(51) Int. Cl. C07K 14/435

(52) U.S. Cl. CPC C07K 14/43518

(57) ABSTRACT

Compositions comprising a porous major ampullate spidroin protein (MaSp)-based fiber, wherein the fiber is characterized by a BET surface area of at least 100 m²/g are disclosed. Further, articles comprising the compositions and methods for producing same are disclosed.

### CROSS-REFERENCE TO RELATED APPLICATIONS

This application claims the benefit of priority from PCT International Patent Application No. PCT/IL2020/050752 filed on Jul. 5, 2020, and from U.S. Provisional Patent Application No. 63/134,343 filed on Jan. 6, 2021. The contents of the above documents are incorporated by reference in their entirety as if fully set forth herein.

### FIELD OF THE INVENTION

The present invention, in some embodiments thereof, is directed to compositions comprising MaSp (major ampullate spidroin) protein-based fibers, preparation and methods of using same.

### BACKGROUND OF THE INVENTION

Dragline spider silk is known in the art as the silk used by the orb-web weaving spiders to construct the frame and radii of their webs as well a lifeline when they fall or escape danger. To be able to perform these tasks, the dragline fiber displays a remarkably high toughness due to combination of high elasticity and strength, which places it as the toughest fiber, whether natural or man-made. For instance, dragline is six times as strong as high-tensile steel in its diameter and three times tougher than Kevlar that is one of the strongest synthetic fibers ever made. Dragline silk consists of two main polypeptides, mostly referred to as major ampullate spidroin (MaSp) 1 and 2, and also to ADF-3 and ADF-4 in Araneus diadematus. These proteins have apparent molecular masses in the range of 200-720 kDa, depending on sample age and conditions of analysis. The known dragline silk spidroins are composed of highly iterated blocks of alternating alanine-rich segments, forming crystalline β-sheets in the fiber, and glycine-rich segments which are more flexible and mainly lack ordered structure. The C-terminal region is non-repetitive, highly conserved between species, and adopts α-helical conformation. The N-terminal region of dragline silk proteins was also found to be highly conserved between different spidroins, and also between different spider species. Numerous attempts have been made to synthetically create spider silk, such as through genetic engineering using bacteria, yeast, plants and mammalian cells in tissue culture and even transgenic goats. There is an unmet need for improved compositions and methods for producing fibers with mechanical properties similar to the natural spider silk.

### SUMMARY OF THE INVENTION

According to one aspect, the present invention provides a composition comprising a porous major ampullate spidroin protein (MaSp)-based fiber, wherein the fiber is characterized by a BET surface area of at least 100 m²/g, and a residual amount of an organic solvent.

In some embodiments, the MaSp based fiber is in the form of particles having a size in the range of 0.5 μm to 2 μm.

In some embodiments, the organic solvent is capable of forming an azeotrope with water.

In some embodiments, the organic solvent comprises t-butanol.

In some embodiments, the composition further comprises additional agent.

In some embodiments, a w/w concentration of the additional agent within the composition is between 1 and 80%.

In some embodiments, a w/w ratio between the additional agent and the porous MaSp based fiber is from 100:1 to 1:10.

In some embodiments, a release rate of the additional agent from the composition is reduced by at least 10%, compared to a control.

In some embodiments, the composition further comprises a thermoplastic polymer selected from polyester, a polyamide, a polyol, a polyurethane, polyethylene, Nylon, polyolefine, a polyacrylate, a polycarbonate, polylactic acid (PLA) or a copolymer thereof, polycaprolactone (PCL), rubber, cellulose, or any combination thereof.

In some embodiments, the weight per weight (w/w) ratio of the MaSp-based fiber to the polymer is between 0.01:1 and 1:1

In some embodiments, the composition comprises between 0.01% to 50% (w/w) of the MaSp-based fiber.

In some embodiments, a tensile strength of the composition is enhanced by at least 20% compared to a control.

In another aspect, there is a provided a method for obtaining a dried major ampullate spidroin protein (MaSp)-based fiber, the method comprising: a. mixing a MaSp-based fiber with a liquid comprising an organic solvent to obtain a mixture; and b. providing the mixture under conditions suitable for substantially removing the liquid from the mixture, thereby obtaining the dried MaSp-based fiber, wherein the dried MaSp-based fiber is characterized by BET surface area of at least 100 m²/g, and a residual amount of an organic solvent.

In some embodiments, the liquid optionally comprises an aqueous solvent.

In some embodiments, the organic solvent is capable of forming azeotrope with water.

In some embodiments, the organic solvent comprises t-butanol.

In another aspect, there is a provided a dried porous major ampullate spidroin protein (MaSp)-based fiber obtained by the method of the present invention.

In some embodiments, the dried MaSp-based fiber is the MaSp-based fiber of the composition of the present invention.

In some embodiments, the dried MaSp-based fiber is, characterized by any of: (i) an improved loading capacity of an additional agent; (ii) sustained release profile.

In some embodiments, the improved loading capacity comprises a w/w ratio between the additional agent and the porous MaSp based fiber is from 100:1 to 1:10.

In another aspect, there is an article comprising the composition of the present invention or the dried MaSp-based fiber of the present invention.

In some embodiments, the article is in the form of reinforced plastics, a bottle, a container, a package, a cable, a tube, a film, a rope, a thread, or a textile.

In some embodiments, the article is characterized by at least one improved mechanical property as compared to the property of the control article, wherein the property is selected from the group consisting of: Young's modulus, tensile strength, fracture strain, yield point, toughness, work to failure, impact strength, tear strength, flexural modulus, flexural strain and stress at a specific percentage elongation, abrasion, UV-resistance and gas permeability.

In some embodiments, the article further comprises a carrier.

In another aspect, there is a method for supplementing a subject with an additional agent, comprising administering to the subject the article of the present invention, thereby supplementing the subject with the additional agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1E are SEM images of porous MaSp-based fibers lyophilized from t-butanol suspension (FIG. 1A, FIG. 1C and FIG. 1E) and lyophilized from H₂O suspension (FIG. 1B and FIG. 1D) at different magnification;

FIG. 2 is a graph presenting the stress-strain curves of polyurethane (PU) (E394POTA) enriched with 15% SVXE lyophilized in t-butanol and in water, compared to control (pristine polyurethane (PU) (E394POTA));

FIGS. 3A-3B are graphs showing the release profile of hyaluronic acid (HA) from SVX-E lyophilized with t-butanol (FIG. 3A) and SVX-E lyophilized with water (FIG. 3B); and

FIGS. 4A-4B are graphs showing the release profile of glycolic acid (GA) from SVX-E lyophilized with t-butanol (FIG. 4A) and SVX-E lyophilized with water (FIG. 4B).

FIG. 5 presents differential scanning calorimetry (DSC) curve of the spider silk protein expressed in bacteria (SVX-E);

FIG. 6 presents differential scanning calorimetry (DSC) curves of the SVX-E at a temperature increase of 25° C.-280° C. (curve 1), cooling back to 50° C. (curve 2), and new increase to 350° C. (curve 3);

FIGS. 7A-7B are thermogravimetric analysis (TGA) curves of the spider silk protein expressed in bacteria (SVX-E).

### DETAILED DESCRIPTION OF THE INVENTION

According to some embodiments, the present invention provides a highly porous major ampullate spidroin protein (MaSp)-based fiber.

According to some embodiments, the present invention provides a composition comprising a porous major ampullate spidroin protein (MaSp)-based fiber, wherein the fiber is characterized by a BET surface area of at least 100 m²/g, and a residual amount of an organic solvent.

The present invention is based, in-part, on the surprising findings that a tensile strength of a composition comprising a porous MaSp-based fiber lyophilized with an organic solvent and enriched with a thermoplastic polymer is enhanced by at least 20% compared to a control. The present invention is based, in-part, on the surprising findings that a tensile strength of a composition comprising a porous MaSp-based fiber lyophilized with t-butanol and enriched with a thermoplastic polymer, is enhanced by at least 20% compared to a to MaSp-based fiber lyophilized with water.

The present invention is based, in-part, on the surprising findings that a composition comprising a porous MaSp-based fiber lyophilized with an organic solvent is characterized by a larger BET surface area, compared to the BET surface area of a porous MaSp-based fiber lyophilized with water. The present invention is based, in-part, on the surprising findings that a composition comprising a porous MaSp-based fiber lyophilized with t-butanol is characterized by a larger BET surface area, compared to the BET surface area of a porous MaSp-based fiber lyophilized with water.

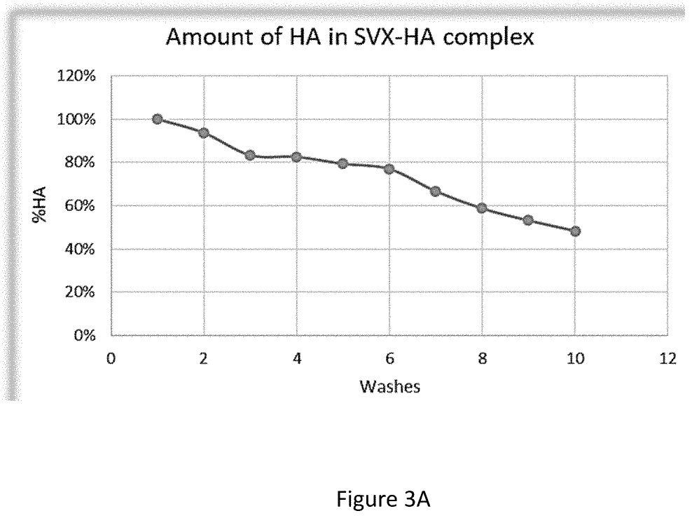

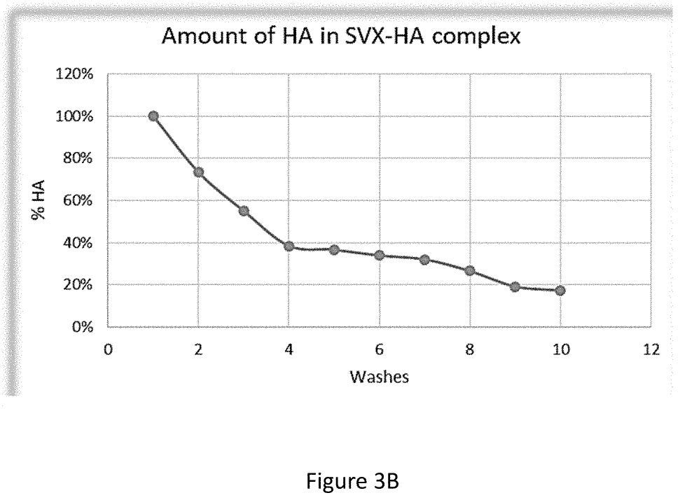

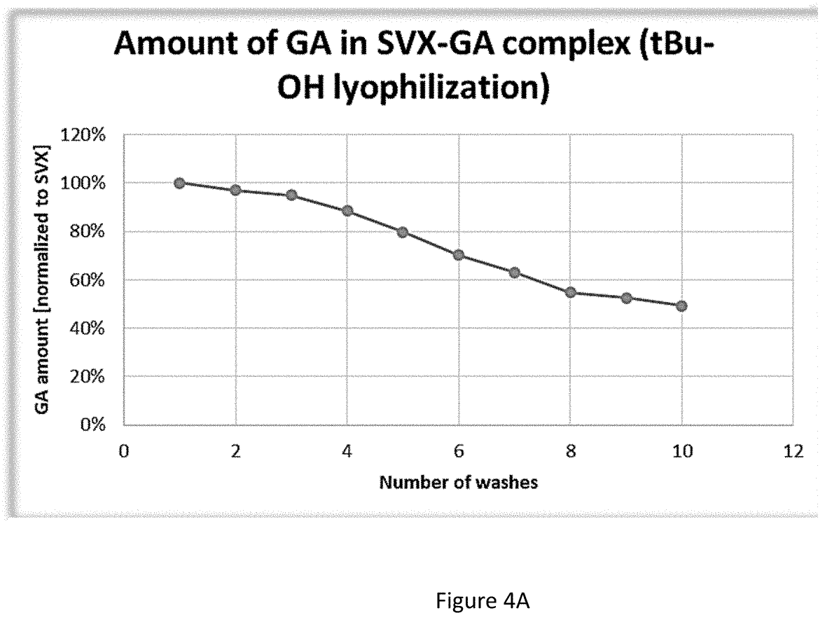

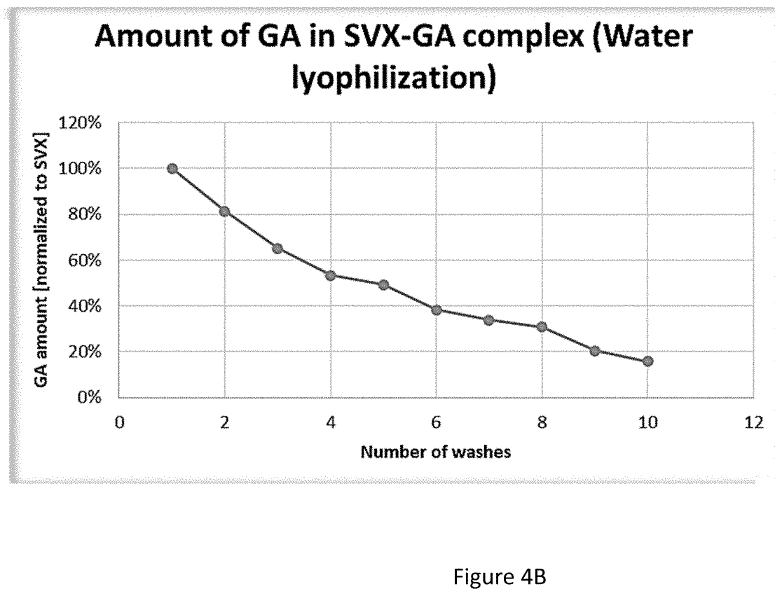

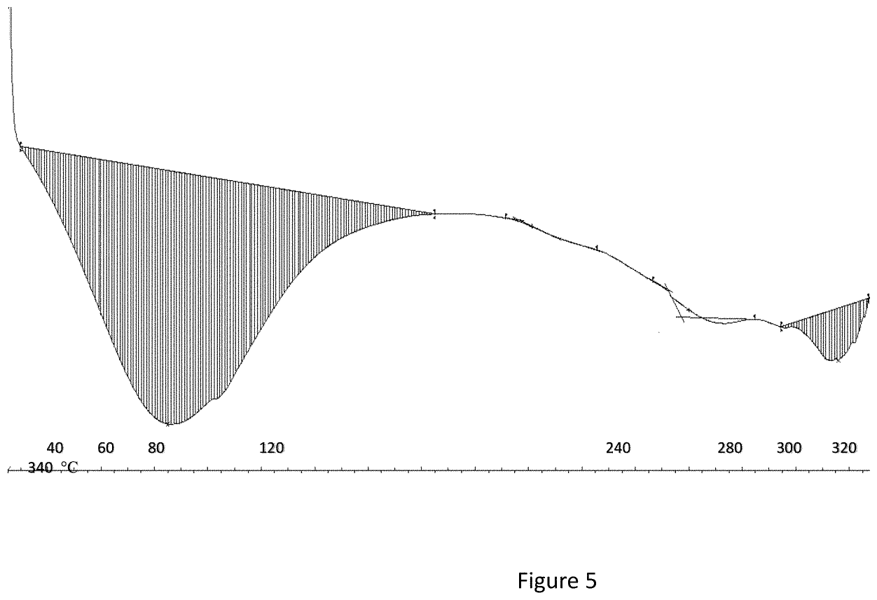

The present invention is based, in-part, on the surprising findings that a composition comprising a porous MaSp-based fiber dried from an organic solvent and enriched with an additional agent, exhibits a slower release rate of the additional agent, compared to a control. The present invention is based, in-part, on the surprising findings that a composition comprising a porous MaSp-based fiber dried from t-butanol and enriched with an additional agent (such as hyaluronic acid or glycolic acid), exhibits a slower release rate of the additional agent, compared to MaSp-based fiber dried from water and enriched with the additional agent. The present invention is based, in-part, on the surprising findings that a composition comprising a porous MaSp-based fiber lyophilized with an organic solvent and enriched with an additional agent, exhibits a slower release rate of the additional agent, compared to a control. The present invention is based, in-part, on the surprising findings that a composition comprising a porous MaSp-based fiber lyophilized with t-butanol and enriched with an additional agent (such as hyaluronic acid or glycolic acid), exhibits a slower release rate of the additional agent, compared to MaSp-based fiber lyophilized with water and enriched with the additional agent (as exemplified in FIGS. 3A-B and FIGS. 4A-B).

### Compositions

According to some embodiments, there is provided a composition comprising a porous major ampullate spidroin protein (MaSp)-based fiber. In some embodiments, the fiber is characterized by a BET surface area of at least 100 m2/g. In some embodiments, the composition comprises a residual amount of an organic solvent. In some embodiments, the fiber of the invention fiber is characterized by a BET surface area of at least 100 m²/g, or of about 180 m²/g or more, and by a residual amount of an organic solvent.

According to some embodiments, there is provided a porous MaSp-based fiber characterized by (i) a residual amount of an organic solvent and (ii) by a BET surface area of at least at least 100 m²/g, at least 150 m²/g, at least 180 m²/g, at least 200 m²/g, at least 210 m²/g, at least 250 m²/g, at least 300 m²/g, at least 350 m²/g, at least 400 m²/g, at least 450 m²/g, at least 500 m²/g, at least 800 m²/g, at least 1000 m²/g, at least 1500 m²/g, at least 2000 m²/g, at least 2500 m²/g, or at least 5000 m²/g, including any value therebetween. Each possibility represents a separate embodiment of the invention.

In some embodiments, there is provided a porous MaSp-based fiber characterized by a BET surface area between 100 and 5500 m²/g, between 100 and 5000 m²/g, between 100 and 2500 m²/g, between 100 and 2000 m²/g, between 100 and 1000 m²/g, between 100 and 500 m²/g, between 100 and 250 m²/g, between 100 and 200 m²/g, between 120 and 5500 m²/g, between 120 and 5000 m²/g, between 120 and 2500 m²/g, between 120 and 2000 m²/g, between 120 and 1000 m²/g, between 120 and 500 m²/g, between 120 and 250 m²/g, between 120 and 200 m²/g, between 150 and 5500 m²/g, between 150 and 5000 m²/g, between 150 and 2500 m²/g, between 150 and 2000 m²/g, between 150 and 1000 m²/g, between 150 and 500 m²/g, between 150 and 250 m²/g, or between 150 and 200 m²/g, including any range therebetween. Each possibility represents a separate embodiment of the invention. In some embodiments, the porous MaSp-based fiber is substantially devoid of an organic solvent and/or of water (e.g. devoid of trace amounts of an organic solvent and/or water).

In one aspect of the invention, there is provided a porous MaSp-based fiber, wherein the porous MaSp-based fiber is characterized by (i) a BET surface area of at least 100 m²/g, at least 150 m²/g, at least 180 m²/g, at least 200 m²/g, at least 210 m²/g, at least 250 m²/g, at least 300 m²/g, at least 350 m²/g, at least 400 m²/g, at least 450 m²/g, at least 500 m²/g, at least 800 m²/g, at least 1000 m²/g, at least 1500 m²/g, at least 2000 m²/g, at least 2500 m²/g, or at least 5000 m²/g, including any value therebetween; (ii) a residual amount of an organic solvent (e.g. t-butanol), as described herein; and/or a water content as described herein (e.g. of less than 100 ppm).

In one aspect of the invention, there is provided a porous MaSp-based fiber, wherein the porous MaSp-based fiber is characterized by (i) a BET surface area of at least 100 m²/g, at least 150 m²/g, at least 180 m²/g, at least 200 m²/g, at least 210 m²/g, at least 250 m²/g, at least 300 m²/g, at least 350 m²/g, at least 400 m²/g, at least 450 m²/g, at least 500 m²/g, at least 800 m²/g, at least 1000 m²/g, at least 1500 m²/g, at least 2000 m²/g, at least 2500 m²/g, or at least 5000 m²/g, including any value therebetween; (ii) a DSC pattern (and/or T_{d}) as described herein; and by (iii) a residual amount of an organic solvent (e.g. tert-butanol), as described herein; and/or a water content as described herein (e.g. of less than 100 ppm). In some embodiments, the porous MaSp-based fiber comprises a plurality of MaSp-based polymers, as described herein. In some embodiments, the porous MaSp-based fiber is in a form of particles. In some embodiments, the porous MaSp-based fiber is in a form of particles within the composition of the invention, wherein the characterized by

In some embodiments, the porous MaSp-based fiber and/or the composition of the invention comprising thereof is further characterized by an improved property, compared to a control, wherein the improved property is one or more of: loading capacity of an additional agent; increased release time of the additional agent encapsulated therewithin; and/or enhanced mechanical strength (e.g. tensile strength).

According to some embodiments, there is provided a composition comprising a porous MaSp-based fiber in the form of particles. In some embodiments, the particles are referred to herein as “porous particles”. In some embodiments, the particles have a size (average particle size) in the range of 0.5 μm to 2 μm, 0.7 μm to 1.5 μm, 0.8 μm to 1.5 μm, 0.9 μm to 1.5 μm, 0.5 μm to 1 μm, 0.7 μm to 1 μm, 0.8 μm to 1 μm, 0.9 μm to 1 μm, 0.5 μm to 1.3 μm, 1.0 μm to 2 μm, 1.0 μm to 2 μm, 1.0 μm to 2 μm, 0.5 μm to 1.2 μm, 0.7 μm to 1.3 μm, 0.7 μm to 1.2 μm, or 0.9 μm to 1.2 μm, including any range therebetween. Each possibility represents a separate embodiment of the invention. In some embodiments, particle size refers to an average particle size within an aqueous solvent (e.g. aqueous dispersion), as measured by laser diffraction (see Examples section). In some embodiments, particle size refers to a number average particle size. One of ordinary skills in the art will appreciate that the average particle size (e.g. number average particle size) can be measured by laser diffraction or by using SEM images. A number average particle size can be calculated according to well-known equations. In some embodiments, particle size refers to a dry particle size (e.g. a size of a particle being substantially devoid of an outer shell comprising water molecules, as measured for example by SEM).

In some embodiments, the particle has spherical shape, elliptical shape, and/or a cylindrical shape. In some embodiments, the particle has a length dimension (e.g. along a longitudinal axis of the particle) in the range of 0.5 μm to 2 μm, 0.7 μm to 1.5 μm, 0.8 μm to 1.5 μm, 0.9 μm to 1.5 μm, 0.5 μm to 1 μm, 0.7 μm to 1 pm, 0.8 μm to 1 μm, 0.9 μm to 1 μm, 0.5 μm to 1.3 μm, 0.5 μm to 1.2 μm, 0.7 μm to 1.3 μm, 0.7 μm to 1.2 μm, or 0.9 μm to 1.2 μm, including any range therebetween. In some embodiments, the particle has a width dimension (e.g. perpendicular to a longitudinal axis of the particle) in the range of 0.01 μm to 0.5 μm, 0.01 μm to 0.05 μm, 0.05 μm to 0.1 μm, 0.1 μm to 0.2 μm, 0.2 μm to 0.3 μm, 0.3 μm to 0.4 μm, 0.4 μm to 0.5 μm, including any range therebetween.

In some embodiments, the porous MaSp-based fiber in the form of a plurality of aggregated particles within the composition of the invention. In some embodiments, the porous MaSp-based fiber in the form of a plurality of distinct particles within the composition of the invention. In some embodiments, at least 50%, at least 60%, at least 70%, at least 80%, at least 90%, at least 95%, at least 99%, at least 99.9% by weight of the porous MaSp-based fiber including any range between, is in a form of plurality of particles, wherein the particles are as described herein. In some embodiments, at least 50%, at least 60%, at least 70%, at least 80%, at least 90%, at least 95%, at least 99%, at least 99.9% by weight of the protein content of the composition including any range between, is in a form of plurality of particles.

In some embodiments, the MaSp-based fiber comprises or consists of an insoluble MaSp-based polymer. In some embodiments, the insoluble MaSp-based polymer is in the form of particles. In some embodiments, the insoluble MaSp-based polymer is insoluble in organic solvents. In some embodiments, the insoluble MaSp-based polymer is insoluble in an aqueous solution. As used herein, the terms “MaSp-based polymer” and “MaSp-based fiber” are used herein interchangeably.

As used herein, the term “insoluble” refers to a material that, when exposed to an excess of solvent, does not dissolve, but may disperse to varying degrees. In some embodiments the term “insoluble” refers to a material that is less than 10%, less than 5%, less than 2%, or less than 1% soluble in a solvent. In some embodiments, “insoluble” refers to a material that can be partially dissolved in a solvent only at a concentration of less than 0.01% by weight. Solvents according to the present invention include organic solvents and aqueous solutions. In some embodiments, the solvent comprises an aqueous surfactant solution. In some embodiments, the solvent comprises urea aqueous solution.

In some embodiments, the MaSp-based fiber is characterized by a defined differential scanning calorimetry (DSC) pattern. In some embodiments, by “DSC pattern” it is meant to refer to the position of the peaks. In some embodiments, by “peak” it is meant to refer to exothermic peak. Herein throughout, “the position of the peaks” or “peak position” refers to the peaks along the temperature axis in a thermogram pattern, and, in some embodiments, may refers to the peak position at any peak intensity. One skilled in the art will appreciate that the data obtained in DSC measurements depend, in part, on the instrument used and the environmental conditions at the time measurements are carried out (e.g., humidity).

In some embodiments, the MaSp-based fiber is characterized by a DSC pattern exhibiting at least an endothermic peak in the range of from 200° C. to 280° C. In some embodiments, the disclosed composition is characterized by a DSC pattern exhibiting at least an endothermic peak in the range of from 200° C. to 270° C., 200° C. to 260° C., 200° C. to 250° C., 210° C. to 280° C., 212° C. to 280° C., 215° C. to 280° C., 216° C. to 280° C., 220° C. to 280° C., 210° C. to 250° C., 212° C. to 250° C., 215° C. to 250° C., 216° C. to 250° C., 220° C. to 250° C., 210° C. to 245° C., 210° C. to 242° C., or 215° C. to 245° C., including any range therebetween. Each possibility represents a separate embodiment of the invention.

In some embodiments, the MaSp-based fiber is further characterized by a DSC pattern exhibiting at least an endothermic peak between 280° C. and 350° C., between 290° C. and 350° C., between 300° C. and 350° C., between 310° C. and 350° C., between 280° C. and 330° C., between 290° C. and 330° C., between 300° C. and 330° C., between 310° C. and 330° C., or between 320° C. and 330° C., including any range therebetween. Each possibility represents a separate embodiment of the invention.

In some embodiments, the MaSp-based fiber is characterized by a glass transition temperature (T_{g}) between 200° C. and 250° C., between 210° C. and 250° C., between 220° C. and 250° C., between 230° C. and 250° C., between 200° C. and 240° C., between 210° C. and 240° C., between 220° C. and 240° C., between 230° C. and 240° C., between 200° C. and 230° C., or between 210° C. and 230° C., as determined by DSC, including any range therebetween. Each possibility represents a separate embodiment of the invention.

In some embodiments, the MaSp-based fiber is characterized by a T_{g} between 260° C. and 320° C., between 270° C. and 320° C., between 280° C. and 320° C., between 290° C. and 320° C., between 260° C. and 310° C., between 270° C. and 310° C., between 280° C. and 310° C., or between 290° C. and 310° C., as determined by DSC, including any range therebetween. Each possibility represents a separate embodiment of the invention.

In some embodiments, the MaSp-based fiber is characterized by a DSC pattern exhibiting at least an endothermic peak with at least 5° C. to 100° C., at least 10° C. to 100° C., at least 15° C. to 100° C., at least 12° C. to 100° C., at least 25° C. to 100° C., at least 5° C. to 80° C., at least 10° C. to 80° C., at least 15° C. to 80° C., at least 12° C. to 80° C., at least 25° C. to 80° C., at least 5° C. to 50° C., at least 10° C. to 50° C., at least 15° C. to 50° C., at least 12° C. to 50° C., or at least 25° C. to 50° C., lower than the DSC pattern of an corresponding composition comprising a (MaSp)-based fiber. Each possibility represents a separate embodiment of the invention.

In some embodiments, the MaSp-based fiber is devoid of DSC peaks in the range of about −100° C. to about 190° C. In some embodiments, the disclosed compound is devoid of DSC peaks in the range of about −100° C. to about 25° C. In some embodiments, the disclosed composition is characterized by at least a DSC pattern exhibiting devoid of an exothermic peak in the range of 40° C. to 70° C.

In some embodiments, the MaSp-based fiber is devoid of DSC peaks in the range of about −100° C. to about −50° C. In some embodiments, the disclosed compound is devoid of DSC peaks in the range of about −50° C. to about 0° C. In some embodiments, the disclosed compound is devoid of DSC peaks in the range of about −0° C. to about −25° C.

The term “degradation temperature (T_{d})” as used herein, refers to a temperature at which decomposition occurs. Thermal decomposition is a process of extensive chemical species change caused by heat.

As used herein, the term “glass transition temperature (T_{g})” refers to the temperature at which a material undergoes a transition from a rubbery, viscous amorphous liquid (T>T_{g}), to a brittle, glassy amorphous solid (T<T_{g}). This liquid-to-glass transition (or glass transition for short) is a reversible transition. The glass transition temperature (T_{g}) is generally lower than the melting temperature (Tₘ), of the crystalline state of the material, if one exists.

In some embodiments, the MaSp-based fiber is characterized by having an amide peak in the range of 1615 cm-1 to 1635 cm-1, as measured by FTIR analysis. In some embodiments, the disclosed composition is characterized by having an amide peak in the range of 1620 cm-1 to 1635 cm-1, 1620 cm-1 to 1630 cm-1, 1621 cm-1 to 1630 cm-1, or 1620 cm-1 to 1625 cm-1, including ay range therebetween, as measured by FTIR analysis. Each possibility represents a separate embodiment of the invention.

In some embodiments, the MaSp-based fiber is devoid of a peak in the range of 1700 cm-1 to 1800 cm-1, as measured by FTIR analysis.

In one embodiment, the MaSp-based polymer of the invention assembles by self-assembly. By “self-assembly” it is meant that monomers, i.e., the synthetic spider silk protein of the invention, bind each other spontaneously, in an energetically favorable manner, under normal physiologic conditions, or at room temperature, to create the macromolecular structure having the properties described herein. Furthermore, the MaSp-based fiber of the invention are extremely resilient, and once assembled, may withstand extreme chemical assaults, such as solubilization in 10% surfactant solution and boiling for at least 1 hour.

According to some embodiments, there is provided a composition comprising a porous major ampullate spidroin protein (MaSp)-based fiber as described hereinabove and a residual amount of an organic solvent.

In some embodiments, the organic solvent disclosed herein is capable of forming an azeotrope with water.

As used herein, the term “azeotrope” or “azeotropic mixture” refers to a system of two or more components in which the liquid composition and vapor composition are equal at the stated pressure and temperature. In practice, this means that the components of an azeotropic mixture are constant-boiling or essentially constant-boiling and generally cannot be thermodynamically separated during a phase change. The vapor composition formed by boiling or evaporation of an azeotropic mixture is identical, or substantially identical, to the original liquid composition. Solvents capable of forming an azeotrope with water are well known and documented in the art and will become apparent to those skilled in the art.

In some embodiments, the organic solvent is selected from the group consisting of ethanol, isopropyl alcohol, t-butanol, 2-butanol, n-butanol, acetonitrile, ethyl acetate, DMF, DMSO, THF, TFA, toluene, hexane, heptane, methyl ethyl ketone, diethyl ketone, methyl isobutyl ketone, dioxane, an ether (e.g. diethyl ether) and any combination thereof

In some embodiments, the organic solvent is a Class 3 solvent. In some embodiments, the Class 3 solvent is selected from the group consisting of acetic acid, acetone, anisole, 1-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

In some embodiments, the composition and/or the MaSp-based fiber of the invention comprises a residual amount of an organic solvent within a pharmaceutically acceptable or a cosmeceutically acceptable range. Thus, the residual amount of the organic solvent disclosed herein, is a pharmaceutically acceptable or a cosmeceutically acceptable amount within the composition and/or fiber of the invention, in some embodiments thereof.

In some embodiments, the composition and/or the MaSp-based fiber of the invention comprises between 0.1 ppm and 100 ppm, 0.5 ppm and 100 ppm, 0.9 ppm and 100 ppm, 1 ppm and 100 ppm, 5 ppm and 100 ppm, 10 ppm and 100 ppm, 20 ppm and 100 ppm, 0.1 ppm and 50 ppm, 0.5 ppm and 50 ppm, 0.9 ppm and 50 ppm, 1 ppm and 50 ppm, 5 ppm and 50 ppm, 10 ppm and 50 ppm, 20 ppm and 50 ppm, 0.1 ppm and 20 ppm, 0.5 ppm and 20 ppm, 0.9 ppm and 20 ppm, 1 ppm and 20 ppm, 5 ppm and 20 ppm, or between 10 ppm and 20 ppm, of the organic solvent, including any range therebetween. Each possibility represents a separate embodiment of the invention.

In some embodiments the composition is substantially devoid of water. In some embodiments the composition is characterized by a water content of less than 100 ppm, less than 70 ppm, less than 50 ppm, less than 40 ppm, less than 20 ppm, less than 10 ppm, less than 10 ppm, less than 5 ppm, less than 1 ppm, less than 0.5 ppm, or less than 0.1 ppm, including any value therebetween. Each possibility represents a separate embodiment of the invention.

In some embodiments, the composition further comprises an additional agent.

In some embodiments, the additional agent is selected from biological agent, a pharmaceutical agent, a nutrient, and a dietary supplement.

The term “biological agent (also designated as biological material)”, as used herein, relates to any substance or material having a biological origin. For example, the term “biological agent” covers cells (including stem cells), proteins, peptides, or nucleic acids (including analogs of nucleic acids). The term “pharmaceutical agent (also designated as pharmaceutical compound)”, as used herein, refers to any biological or chemical substance, which may be used in the treatment, cure, prophylaxis, prevention, or diagnosis of a pathological condition, e.g. a disease or disorder, or which may be used to otherwise enhance the physical, psychical, or mental well-being. Accordingly, the term “pharmaceutical agent” envisaged in the context of the present invention includes any agent with therapeutic, diagnostic, or prophylactic effects, i.e. any therapeutic agent, diagnostic agent, or prophylactic agent.

The pharmaceutical agent may be an agent that affects or participates in tissue growth, cell growth, cell differentiation, an agent that is able to invoke a biological action such as an immune response, or an agent that can play any other role in one or more biological processes.

Non-limiting examples of pharmaceutical agents include but are not limited to an anti-microbial agent, such as an antibacterial agent (e.g. an antibiotic), an anti-viral agent or an anti-fungal agent, an immunosuppressive agent, an anti-inflammatory agent, an anti-allergic agent, an anti-coagulant, an anti-rheumatic agent, an anti-psoriatic agent, a sedative agent, a muscle relaxant, an anti-migraine agent, an anti-depressant, an insect repellent, a growth factor, a hormone, a hormone antagonist, an antibody, an adjuvant, e.g. in combination with an immunological active compound such as an antibody, an antioxidant, a protein, such as a glycoprotein, lipoprotein, or an enzyme (e.g. hyaluronidases), a polysaccharide, a free radical scavenger, a radio-therapeutic agent, a photodynamic therapy agent, a dye (e.g. fluorescent dye), a contrast agent, a disinfectant, a preservative, or any combination thereof.

The pharmaceutical agent may also be a small molecule compound. The term “small molecule compound” refers to a molecule that can act to affect biological processes. Small molecules can include any number of therapeutic agents presently known and used, or can be small molecules synthesized in a library of such molecules for the purpose of screening for biological function(s). The small molecule compound usually has a molecular weight less than about 5,000 daltons (Da), preferably less than about 2,500 Da, more preferably less than 1,000 Da, most preferably less than about 500 Da.

As used herein, a “nutrient” is a chemical that an organism needs to live and grow or a substance used in an organism's metabolism which must be taken in from its environment. Organic nutrients include carbohydrates, fats, proteins (amino acids), and vitamins. Inorganic nutrients are dietary minerals, water, and oxygen. Preferred nutrients are macronutrients such as carbohydrates, amino acids or proteins and micronutrients such as vitamins.

Non-limiting examples of carbohydrates include but are not limited to monosaccharides such as, glyceraldehyde, erythrose, threose, ribose, arabinose, xylose, lyxose, allose, altrose, glucose, mannose, gulose, idose, galactose, talose, dihydroxacetone, erythrulose, ribulose, xylulose, psicose, fructose, sorbose, tagatose or stereoisomers thereof, amino sugars such as galactosamine, glucosamine, sialic acid, N-acetylglucosamine, sulfo sugars such as sulfoquinovose, disaccharides such as sucrose, lactulose, lactose, maltose, trehalose or maltobiose, and oligosacharides such as Fructooligosaccharides (FOS), Galactooligosaccharides (GOS) or Mannan-oligosaccharides (MOS).

The term “dietary supplement” (also designated as food supplement or nutritional supplement), as used herein, refers to a preparation intended to provide nutrients such as vitamins, minerals, fiber, fatty acids or amino acids, that are missing or are not consumed in sufficient quantity in a person's diet.

Non-limiting examples of dietary supplements include, but are not limited to, steroids such as dehydroepiandrosterone (DHEA), pregnenolone, or derivatives thereof, hormones such as melatonin, and other substances such as hydrrazine sulfate, caffeine, catechins, soy isoflavones, glucosamine, coenzyme-Q₁₀, or ephedrine-type alkaloids such as ephedrine, synephrine, norephedrine, or pseudodoephedrine.

The additional agent may be positively or negatively charged. The additional agent may also be electroneutral. Preferably, the additional agent is positively or negatively charged. The terms “positive charge” and “cationic” as well as “negative charge” and “anionic” can be used interchangeably.

In some embodiments, the MaSp-based polymer particles comprise a plurality of pores (i.e. a space or lumen) formed by the intertwisted polymeric chains of the MaSp-based polymer. In some embodiments, the entangled or intertwined MaSp-based polymers form a matrix. In some embodiments, the additional agent fills at least a portion of the pores within the matrix or within the particle. In some embodiments, the additional agent is encapsulated by the plurality of pores.

In some embodiments, the physical interaction is referred to the encapsulation (i.e. entrapment) of the additional agent within a matrix formed by the MaSp-based polymer. In some embodiments, the matrix is bound or in contact with the additional agent. In some embodiments, the (MaSp)-based fiber is bound to the additional agent.

In some embodiments, the additional agent is stably encapsulated within the plurality of pores of the particle. In some embodiments, the stably encapsulated additional agent is characterized by a gradual release profile (e.g. on the application site or in a solution). In some embodiments, the particle encapsulating the additional agent substantially prevents a rapid release of the additional agent therefrom.

In some embodiments, the stably encapsulated additional agent is characterized by a prolonged release time compared to a control, wherein the control is as described herein.

As used herein, the term “stably encapsulated” refers to the ability of the composition to substantially prevent a rapid release of the additional agent therefrom.

In some embodiments, a w/w concentration of the additional agent within the composition is between 1% and 80%, 3% and 80%, 5% and 80%, 10% and 80%, 20% and 80%, 30% and 80%, 50% and 80%, 1% and 60%, 3% and 60%, 5% and 60%, 10% and 60%, 20% and 60%, 30% and 60%, 50% and 60%, 1% and 50%, 3% and 50%, 5% and 50%, 10% and 50%, 20% and 50 %, between 30% and 80%, or between 50 and 99.9%, including any range therebetween. Each possibility represents a separate embodiment of the invention.

In some embodiments, substantially comprises at least 50%, at least 60%, at least 70%, at least 75%, at least 80%, at least 85%, at least 90%, at least 95%, at least 97%, at least 99%, by weight of the additional agent. In some embodiments, substantially comprises at least 50%, at least 60%, at least 70%, at least 75%, at least 80%, at least 85%, at least 90%, at least 95%, at least 97%, at least 99%, by weight of additional agent is bound to the MaSp-based fiber via a non-covalent bond, via a physical interaction or both. Each possibility represents a separate embodiment of the invention. Non-covalent bonds are well-known in the art and include inter alia hydrogen bonds, p-p stacking, Van der Waals interactions, etc.

In some embodiments, the physical interaction is referred to the encapsulation (i.e. entrapment) of the additional agent within a matrix formed by the MaSp-based polymer. In some embodiments, the matrix is bound or in contact with the additional agent.

In some embodiments, the additional agent is in contact with or bound to the MaSp-based fiber. In some embodiments, the polymer enriched with the additional agent is physically bound to the MaSp-based fiber. In some embodiments, the additional agent fills at least a portion of the pores on or within the MaSp-based fiber. In some embodiments, the additional agent is encapsulated by the MaSp-based fiber. In some embodiments, the additional agent is in contact with or bound to the fibrils. In some embodiments, the additional agent is encapsulated by an intertwisted structure (also used herein as a “matrix”) of the particle. In some embodiments, the additional agent is encapsulated by the particle. In some embodiments, the additional agent is incorporated within the MaSp-based fiber. In some embodiments, the additional agent is embedded within the MaSp-based fiber. In some embodiments, the additional agent is embedded within the matrix. In some embodiments, the matrix is doped by the additional agent. In some embodiments, the additional agent is located within the plurality of pores. In some embodiments, the additional agent is located between the fibrils. In some embodiments, the additional agent is located within a lumen, wherein the lumen is defined by intertwisted fibers of the matrix. In some embodiments, the additional agent is encapsulated by the fibrils. In some embodiments, the additional agent is encapsulated within a lumen (or void space) in each of the fibrils.

In some embodiments, bound is via a non-covalent bond, a physical interaction or both.

In some embodiments, the additional agent fills 20% to 100% of the volume of the pores. In some embodiments, the additional agent fill 55% to 100%, 60% to 100%, 55% to 100%, 70% to 100%, 75% to 100%, 80% to 100%, 85% to 100%, 90% to 100%, 95% to 100%, 50% to 99%, 50% to 98%, 50% to 97%, 50% to 95%, 50% to 90%, 70% to 90%, or 70% to 95% of the volume of the pores, including any range therebetween. Each possibility represents a separate embodiment of the invention.

In some embodiments, the additional agent fills 20% to 100% of the volume (e.g. lumen) of the particle. In some embodiments, the additional agent fill 55% to 100%, 60% to 100%, 55% to 100%, 70% to 100%, 75% to 100%, 80% to 100%, 85% to 100%, 90% to 100%, 95% to 100%, 50% to 99%, 50% to 98%, 50% to 97%, 50% to 95%, 50% to 90%, 70% to 90%, or 70% to 95% of the volume of the particle, including any range therebetween. Each possibility represents a separate embodiment of the invention.

In some embodiments, a w/w ratio of the additional agent to the MaSp-based fiber within the composition of the invention is from 100:1 to 1:10, from 100:1 to 8:1, from 100:1 to 6:1, from 100:1 to 4:1, 100:1 to 1:1, from 100:1 to 10:1, 90:1 to 1:10, from 90:1 to 8:1, from 90:1 to 6:1, from 90:1 to 4:1, 90:1 to 1:1, from 90:1 to 10:1, 50:1 to 1:10, from 50:1 to 8:1, from 50:1 to 6:1, from 50:1 to 4:1, 50:1 to 1:1, from 50:1 to 10:1, 20:1 to 1:10, from 20:1 to 8:1, from 20:1 to 6:1, from 20:1 to 4:1, 20:1 to 1:1, from 20:1 to 10:1, 10:1 to 1:10, from 10:1 to 8:1, from 8:1 to 6:1, from 6:1 to 4:1, from 4:1 to 3:1, from 3:1 to 2:1, from 2:1 to 1:1, from 1:1 to 1:2, from 1:2 to 1:3, from 1:3 to 1:5, from 1:5 to 1:10 including any range therebetween. Each possibility represents a separate embodiment of the invention.

In some embodiments, a w/w ratio of the additional agent to the MaSp-based fiber within the composition is at most 100:1, at most 80:1, at most 40:1, at most 20:1, at most 10:1, at most 6:1, at most 5:1, at most 4:1, at most 3:1, at most 2:1, at most 1:1 including any range therebetween. Each possibility represents a separate embodiment of the invention.

In some embodiments, the release rate of the additional agent from the composition is reduced by at least 10%, compared to a control. In some embodiments, the release rate of the additional agent from the composition is reduced by at least 10%, at least 20%, at least 30%, at least 50%, at least 70%, at least 90%, at least 100%, at least 150%, at least 200%, at least 250%, at least 300%, at least 400%, at least 450%, at least 500%, at least 600%, at least 700%, at least 800%, at least 1000%, including any range or value therebetween. Each possibility represents a separate embodiment of the invention.

In some embodiments, the control is a MaSp-based fiber lyophilized from water. As exemplified hereinbelow (FIGS. 3 and 4), the composition of the invention is characterized by an improved release profile (i.e. reduced release rate) of the additional agent compared to a control (e.g. Masp-based fiber lyophilized from water).

In some embodiments, the release period is prolonged by at least 50%, at least 100%, at least 200%, at least 300%, at least 400%, at least 500%, at least 600%, at least 700%, at least 800%, at least 1000% compared to a control, including any range or value therebetween. Each possibility represents a separate embodiment of the invention.

In some embodiments, the composition further comprises a thermoplastic polymer. As used herein “thermoplastic” refers to a material which becomes softer when heated and hard when cooled. Thermoplastic materials can be cooled and heated several times without any change in their chemical or mechanical properties.

In some embodiments, the thermoplastic polymer selected from polyester, a polyamide, a polyol, a polyurethane, polyethylene, Nylon, polyolefine, a polyacrylate, a polycarbonate, polylactic acid (PLA) or a copolymer thereof, polycaprolactone (PCL), rubber, cellulose, or any combination thereof.

In some embodiments, a weight per weight (w/w) ratio of the MaSp-based fiber to the high melt temperature polymer is between 0.01:1 and 1:1, 0.02:1 and 1:1, 0.05:1 and 1:1, 0.09:1 and 1:1, 0.1:1 and 1:1, 0.5:1 and 1:1, or between 0.9:1 and 1:1, including any range therebetween. Each possibility represents a separate embodiment of the invention.

In some embodiments, the composition comprises 0.01% to 90% (w/w), 0.05% to 90% (w/w), 0.09% to 90% (w/w), 0.1% to 90% (w/w), 0.5% to 90% (w/w), 0.9% to 90% (w/w), 1% to 90% (w/w), 5% to 90% (w/w), 10% to 90% (w/w), 15% to 90% (w/w), 20% to 90% (w/w), 30% to 90% (w/w), 0.01% to 80% (w/w), 0.05% to 80% (w/w), 0.09% to 80% (w/w), 0.1% to 80% (w/w), 0.5% to 80% (w/w), 0.9% to 80% (w/w), 1% to 80% (w/w), 5% to 80% (w/w), 10% to 80% (w/w), 15% to 80% (w/w), 20% to 80% (w/w), 30% to 80% (w/w), 0.01% to 50% (w/w), 0.05% to 50% (w/w), 0.09% to 50% (w/w), 0.1% to 50% (w/w), 0.5% to 50% (w/w), 0.9% to 50% (w/w), 1% to 50% (w/w), 5% to 50% (w/w), 10% to 50% (w/w), 15% to 50% (w/w), 20% to 50% (w/w), 30% to 50% (w/w), 0.01% to 20% (w/w), 0.05% to 20% (w/w), 0.09% to 20% (w/w), 0.1% to 20% (w/w), 0.5% to 20% (w/w), 0.9% to 20% (w/w), 1% to 20% (w/w), 5% to 20% (w/w), 0.01% to 10% (w/w), 0.05% to 10% (w/w), 0.09% to 10% (w/w), 0.1% to 10% (w/w), 0.5% to 10% (w/w), 0.9% to 10% (w/w), or 1% to 10% (w/w), of the MaSp-based fiber, including any range therebetween. Each possibility represents a separate embodiment of the invention.

In some embodiments, one or more properties of the composition, selected from thermal stability, Young's modulus, tensile strength, yield point, abrasion resistance, and stress at elongation, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, a tensile strength of the composition is enhanced by at least 20%, at least 21%, at least 22%, at least 23%, at least 24%, at least 25%, at least 26%, at least 27%, at least 28%, at least 29%, at least 30%, at least 50%, at least 100%, at least 200%, or at least 500% compared to a control, including any value therebetween. Each possibility represents a separate embodiment of the invention.

In some embodiments, the composition is characterized by a Young's modulus in the range of 50 MPa to 170 MPa, 52 MPa to 170 MPa, 60 MPa to 170 MPa, 68 MPa to 170 MPa, 90 MPa to 170 MPa, 100 MPa to 170 MPa, 101 MPa to 170 MPa, 105 MPa to 170 MPa, 101 MPa to 160 MPa, or 105 MPa to 160 MPa, including any range therebetween. Each possibility represents a separate embodiment of the invention.

“Tenacity” or “tensile strength” refers to the amount of weight a filament can bear before breaking. The maximum specific stress that is developed is usually in the filament, yarn or fabric by a tensile test to break the materials. According to specific embodiments, the MaSp-based polymer of the invention has tensile strength of about 100-3000 MPa (MPa=N/mm2), about 300-3000 MPa, about 500-2700 MPa, about 700-2500 MPa, about 900-2300 MPa, about 1100-2000 MPa, about 1200-1800 MPa, about 1300-1700 MPa or about 1400-1600 MPa. More specifically, about 1500 MPa.

“Toughness” refers to the energy needed to break the MaSp-based polymer. This is the area under the stress strain curve, sometimes referred to as “energy to break” or work to rupture. According to particular embodiments, the MaSp-based polymer of the invention a toughness of about 20-1000 MJ/m3, about 50-950 MJ/m3, about 100-900 MJ/m3, about 120-850 MJ/m3, about 150-800 MJ/m3, about 180-700 MJ/m3, about 180-750 MJ/m3, about 250-700 MJ/m3, about 280-600 MJ/m3, about 300-580 MJ/m3, about 310-560 MJ/m3, about 320-540 MJ/m3 or about 350-520 MJ/m3, most specifically about 350-520 MJ/m3.

“Elasticity” refers to the property of a body which tends to recover its original size and shape after deformation. Plasticity, deformation without recovery, is the opposite of elasticity. On a molecular configuration of the MaSp-based polymer, recoverable or elastic deformation is possible by stretching (reorientation) of inter-atomic and inter-molecular structural bonds. Conversely, breaking and re-forming of intermolecular bonds into new stabilized positions causes non-recoverable or plastic deformations.

“Extension” refers to an increase in length expressed as a percentage or fraction of the initial length.

By “fineness” is meant the mean diameter of a MaSp-based polymer or filament (e.g., a biofilament), which is usually expressed in microns (micrometers).

Without being bound to any particular theory, it is postulated that the high porosity of the MaSp-based fiber disclosed herein, predetermines several advantageous properties of the fiber, such as release profile, tensile strength and optionally loading capacity.

According to some aspects, the MaSp-based protein or the MaSp-based polymer which are used herein interchangeably, is in the form of a fiber. A “fiber” as used herein, is meant a fine cord of fibrous material composed of two or more filaments twisted together. By “filament” is meant a slender, elongated, threadlike object or structure of indefinite length, ranging from microscopic length to lengths of a mile or greater. Specifically, the synthetic spider silk filament is microscopic, and is proteinaceous. By “biofilament” is meant a filament created from a protein, including recombinantly produced spider silk protein. In some embodiments, the term “fiber” does not encompass unstructured aggregates or precipitates.

In some embodiments, the MaSp-based fiber comprises a plurality of MaSp-based polymers. In some embodiments, the plurality of MaSp-based polymers comprises polymers having a different chemical composition and/or a different molecular weight (MW). In some embodiments, the plurality of MaSp-based polymers comprises polymers having a different number of repetitive regions.

In some embodiments, the composition of the invention substantially comprises a single MaSp-based polymer or the MaSp-based fiber, as described herein. In some embodiments, the composition of the invention substantially comprises a single additional agent or a plurality (e.g. 2, 3, 4, 5, 10, etc.) of additional agents. In some embodiments, the MaSp-based polymer or the MaSp-based fiber is substantially devoid of an additional non-MaSp-based protein. In one embodiment, the MaSp-based polymer or the MaSp-based fiber is substantially devoid of an additional polymer (e.g. a synthetic polymer, a non-MaSp-based peptide, non-MaSp-based protein).

In some embodiments, at least 80%, at least 90%, at least 95%, at least 97%, at least 99%, at least 99.5%, at least 99.9%, or between 80 and 99%, between 90 and 99.9% nby weight of the composition of the invention including any range between, is composed of the MaSp-based fiber of the invention (e.g. a single MaSp-based fiber specie) and optionally of the one or more additional agent, as described herein. In some embodiments, at least 80%, at least 90%, at least 95%, at least 97%, at least 99%, at least 99.5%, at least 99.9% by weight of the protein content within the composition of the invention including any range between, is composed of the MaSp-based fiber of the invention. In some embodiments, at least 80%, at least 90%, at least 95%, at least 97%, at least 99%, at least 99.5%, at least 99.9% by weight of the polymer content within the composition of the invention including any range between, is composed of the MaSp-based fiber of the invention.

In some embodiments, at least 90%, at least 95%, at least 97%, at least 99%, at least 99.5%, at least 99.9%, at least 99.99%, or between 80 and 99%, between 90 and 99.9% by weight of the MaSp-based fiber of the invention including any range between, is composed of the MaSp-based polymer of the invention (e.g. a single amino acid sequence, or a plurality of amino acid sequences, as described herein).

In some embodiments, the fiber of the proteins is characterized by size of at least one dimension thereof (e.g., diameter, length). For example, and without limitation, the diameter of the fiber is between 10 nm-1μm, 20-100 nm, or 10-50 nm.

In one embodiment, the MaSp-based fiber is composed of a plurality of MaSp-based polymers (e.g., each of the MaSp-based polymers is selected from a peptide, a polyamino acid, or a polypeptide having the same or different amino acid sequence). In one embodiment, a plurality of MaSp-based polymers have the same amino acid sequence, being selected from the SEQ ID Nos. described herein. In one embodiment, a plurality of MaSp-based polymers are arranged in a nanofibril. In one embodiment, a plurality of nanofibrils are arranged in a fiber or make-up a fiber. In one embodiment, a monomer or a nanofibril within the MaSp-based fiber has a diameter of 4 to 16 nm. In one embodiment, a monomer or a nanofibril within the MaSp-based fiber has a diameter of 6 to 14 nm. In one embodiment, a monomer or a nanofibril within the MaSp-based fiber has a diameter of 8 to 12 nm. In some embodiments, the MaSp-based fiber comprises a plurality of fibrils (e.g. nanofibrils). In some embodiments, a fiber having a mutated amino acid sequence is substantially devoid of nanofibrils. In some embodiments, a fiber having a mutated amino acid sequence is in a form of a non-porous particle.

In some embodiments, the nanofibrils have a diameter of e.g., 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 40 nm, about 42 nm, about 44 nm, about 46 nm, about 48 nm, or about 50 nm, including any value or range therebetween. In one embodiment, the nanofibrils have a diameter of 3-7 nm. In one embodiment, the nanofibrils have a diameter of 4-6 nm.

In one embodiment, the MaSp-based fiber has a diameter of 70 to 450 nm. In one embodiment, the MaSp-based fiber has a diameter of 80 to 350 nm. In one embodiment, the MaSp-based fiber has a diameter of 80 to 300 nm. In one embodiment, the MaSp-based fiber has a diameter of 150 to 250 nm. In one embodiment, the MaSp-based fiber or the MaSp-based polymer is arranged as a coil. In one embodiment, a single fiber or one the MaSp-based polymer is arranged as a coil. In one embodiment, a coil has a diameter of 5 to 800 micrometers. In one embodiment, a coil has a diameter of 5 to 500 micrometers. In one embodiment, a coil has a diameter of 5 to 30 micrometers. In one embodiment, a coil has a diameter of 5 to 20 micrometers. In one embodiment, the MaSp-based fiber or the MaSp-based polymer has a length of 5 to 800 micrometers. In one embodiment, the MaSp-based fiber or the MaSp-based polymer has a length of 30 to 300 micrometers. In some embodiments, the length of the MaSp-based fiber is between 1-200 μm, 10-100 μm, 100 to 500 μm or 200-500 μm, including any range therebetween.

In some embodiments, the MaSp-based fiber comprises a plurality of pores. In some embodiments, the MaSp-based fiber is in a form of a particle, as described herein. In some embodiments, the composition comprises a plurality of MaSp-based fibers. In some embodiments, the plurality of MaSp-based fibers comprises fibers having a different chemical composition. In some embodiments, the plurality of MaSp-based fibers are in a form of particles having different size and/or different structure. In some embodiments, the plurality of MaSp-based fibers are in a form of particles having different porosity (expressed by BET surface area). Porosity may be measured using BET surface analysis, as described, for example, in S. Brunauer, P. H. Emmett, and E. Teller, J. Am. Chem. Soc., 1938, 60, 309, which is incorporated herein by reference in its entirety.

In some embodiments, the MaSp-based fiber of the invention is characterized by a porous structure. In some embodiments, the MaSp-based fiber is a porous fiber. In some embodiments, the MaSp-based fiber of the invention is in a form of porous particles having a mesh-like or a sponge-like structure, wherein the particle size is as described herein. In some embodiments, the porous particles are in a form of a matrix, as described herein (e.g. comprising a plurality of mesh-like intertwisted MaSp-polymers defining a plurality of void spaces or lumens). In some embodiments, the MaSp-based fiber of the invention comprises a plurality of distinct porous particles, wherein the porous particles are as described herein (e.g. having a median size in the range of 0.5 μm to 1.5 μm). Whereas the MaSp-based fibers of the control (e.g. dried from an aqueous solution) are in a form of substantially less porous fibers, having a size of more than 2 μm, and are substantially uniform thread-like fibers, e.g. devoid of distinct porous particles.

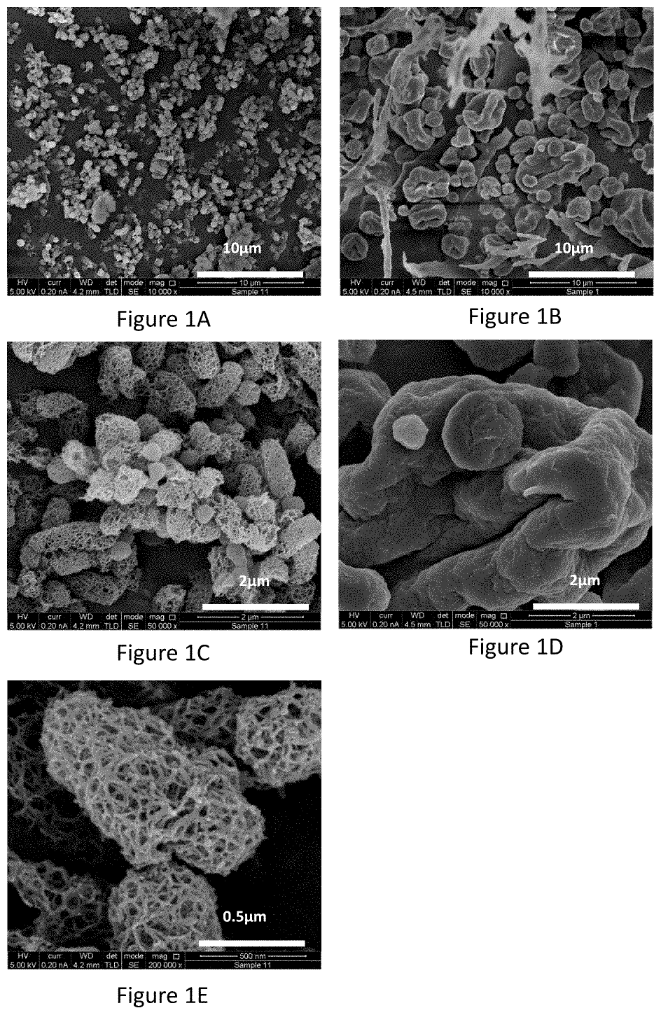

Exemplary structure of the porous fibers of the invention versus non-porous analogous MaSp-based fibers are represented in FIG. 1.

In some embodiments, the MaSp-based fiber of the invention is characterized by a pore size of at least 10 nm, at least 20 nm, at least 30 nm, at least 40 nm, at least 50 nm, at least 60 nm, at least 80 nm, at least 100 nm, including any range between. In some embodiments, the MaSp-based fiber of the invention is characterized by a pore size of between 20 and 80 nm, between 20 and 60 nm, including any range between. In some embodiments, the pore size values described herein are median values. In some embodiments, at least 60%, at least 70%, at least 80%, at least 85%, at least 90%, at least 95% of the pore are characterized by a pore size as described herein.

Without being bound to any theory or mechanism, it is postulated that MaSp-based fibers of the control (e.g. dried from an aqueous solution) have a pore size of greater than 500 nm, greater than 1 μm or even more.

In some embodiments, the porous structure or the porous MaSp-based fiber is characterized by a porosity of at least 30% (e.g., from 30 to 99%). In some embodiments, the porous structure is characterized by a porosity of at least 50% (e.g., from 50 to 99%). In some embodiments, the porous structure is characterized by a porosity of at least 60% (e.g., from 60 to 99%). In some embodiments, the porous structure is characterized by a porosity of at least 70% (e.g., from 70 to 99%). In some embodiments, the porous structure is characterized by a porosity of at least 80% (e.g., from 80 to 99%). In some embodiments, the porous structure is characterized by a porosity of at least 90% (e.g., from 90 to 99%). In some embodiments, the porous structure is characterized by a porosity of about 90%.

Herein, the term “porosity” refers to a percentage of the volume of a substance (e.g., a “sponge-like” material) which consists of voids. In another embodiment, porosity is measured according to voids within the surface area divided to the entire surface area (porous and non-porous).

In some embodiments, the porous structure of the disclosed MaSp-based fibers allows absorbing an additional agent or a polymer efficiently within the plurality of pores of the MaSp-based fiber of the invention. It is postulated, that the enhanced porosity of the MaSp-based fibers is responsible for an increased retention of the additional agent therewithin, compared to a control (e.g. a similar fiber dried form an aqueous solution) and having substantially lower void space and/or BET value (as exemplified in FIG. 1). That is, and without being bound by any particular theory, this surprising discovery can be explained in view of the disclosed fiber structure and its porosity which is in sharp distinction from native spider silk found in nature or from similar MaSp-based fiber (e.g. having the same amino acid sequence) dried form an aqueous solution and characterized by “collapsed” structure and having substantially less porous structure, compared to the fiber of the invention. As exemplified in FIG. 1, the fiber lyophilized from an aqueous solution is substantially in a form of a film.

In some embodiments, the MaSp-based fibers of the invention have at least 20%, at least 50%, at least 100%, at least 200%, at least 500%, at least 1000%, at least 2000% greater BET value, compared to a control, wherein the control is as described herein.

In some embodiments, the porous MaSp-based fiber is in a form of a particle, as described herein. In some embodiments, the particle is a porous particle. In some embodiments, the particle is substantially non-aggregated particle. In some embodiments, the particle comprising a plurality of pores (i.e. a space or lumen) formed by the intertwisted polymeric chains of the MaSp-based polymer. In some embodiments, the entangled or intertwined MaSp-based polymers form a matrix. In some embodiments, the additional agent fills at least a portion of the pores within the matrix or within the particle. In some embodiments, the additional agent is encapsulated by the plurality of pores.

### Article

In another aspect of the invention, there is an article comprising the composition of the invention or the dried MaSp-based fiber of the invention.

In another aspect of the invention, there is an article comprising the composition of the invention or the lyophilized MaSp-based fiber of the invention.

In some embodiments, the article is in the form of reinforced plastics, a bottle, a container, a package, a cable, a tube, a film, a rope, a thread, or a textile.

In some embodiments, the article is characterized by at least one improved mechanical property as compared to the property for the article free of the composition, wherein the property is selected from the group consisting of: Young's modulus, tensile strength, fracture strain, yield point, toughness, work to failure, impact strength, tear strength, flexural modulus, flexural strain and stress at a specific percentage elongation, abrasion, UV-resistance and gas permeability.

In some embodiments, the article further comprises a carrier.

In some embodiments, the article is a cosmetic product (e.g. a color cosmetic product, a powder, a face cleanser). The cosmetic product can be those described in other sections of this specification or those known to a person of skill in the art. Non-limiting examples of products include a moisturizer, a cream, a lotion, a skin softener, a foundation, a night cream, a lipstick, a cleanser, a toner, a sunscreen, a mask, an anti-aging product, a deodorant, an antiperspirant, a perfume, a cologne, etc.

In some embodiments, the carrier is a physiologically suitable carrier. Exemplary physiologically suitable carriers are listed hereinbelow, and additional physiologically suitable carriers are well-known in the art.

In some embodiments, the carrier comprises an emulsifier. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560).

Non-limiting examples of emulsifiers include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, or any combination thereof.

### Method

In another aspect of the invention, there is a method for obtaining a dried major ampullate spidroin protein (MaSp)-based fiber, the method comprising: a. mixing a MaSp-based fiber with a liquid comprising an organic solvent to obtain a mixture; and b. providing the mixture under conditions suitable for substantially removing the liquid from the mixture, thereby obtaining the dried MaSp-based fiber, wherein the dried MaSp-based fiber is characterized by BET surface area of at least 100 m²/g, and optionally by a residual amount of an organic solvent. In some embodiments, the step a and the step b are performed simultaneously or subsequently. In some embodiments, the step a is performed prior to performing the step b.

In some embodiments, the dried MaSp-based fiber is the MaSp-based fiber of the invention, as described herein. In some embodiments, the MaSp-based fiber of the invention is dried by the method disclosed herein.

In another aspect of the invention, there is a method for obtaining a lyophilized major ampullate spidroin protein (MaSp)-based fiber, the method comprising: a. mixing a MaSp-based fiber with the liquid to obtain a mixture; and b. providing the mixture under conditions suitable for lyophilization, thereby obtaining the lyophilized MaSp-based fiber.

In some embodiments, the method is for obtaining a dried major ampullate spidroin protein (MaSp)-based fiber, the method comprises: a. mixing a wet MaSp-based fiber with a liquid comprising an organic solvent to obtain a mixture; and b. providing the mixture under conditions suitable for substantially removing the liquid from the mixture, thereby obtaining the dried MaSp-based fiber. In some embodiments, the method comprises a step of providing a wet MaSp based fiber, wherein providing is performed prior to executing any of the steps a and b. In some embodiments, wet MaSp-based fiber is obtained (e.g. extracted) from any of the expression systems described herein. In some embodiments, wet MaSp-based fiber is isolated from a host cell or from a host organism (also referred to as “expression system”).

In some embodiments, the method comprises: mixing a MaSp-based fiber (e.g. obtained from the expression system and comprising water) with a liquid comprising an organic solvent to obtain a mixture; and providing the mixture under conditions suitable for drying the MaSp-based fiber. In some embodiments, mixing comprises adding the liquid to the wet MaSp-based fibers, or vice versa. In some embodiments, adding is by pouring the liquid on top of the wet MaSp-based fibers. In some embodiments, mixing comprises adding the wet MaSp-based fibers to the liquid.

In some embodiments, the conditions suitable for drying comprise conditions sufficient for removing of at least 99%, at least 99.9%, at least 99.99%, at least 99.999%, or more of the solvent (e.g. the liquid and water content of the wet fibers) from the mixture, thereby obtaining the dried MaSp-based fiber. In some embodiments, the conditions suitable for drying comprise thermal exposure and/or exposure to IR or MW radiation having an intensity sufficient for removing of at least 99%, at least 99.9%, at least 99.99%, at least 99.999% or more of the solvent (e.g. the liquid and water content of the wet fibers) from the mixture. In some embodiments, the conditions suitable for drying comprise thermal exposure and/or exposure to IR or MW radiation for a time period sufficient for removing of at least 99%, at least 99.9%, at least 99.99%, at least 99.999% or more of the solvent (e.g. the liquid and water content of the wet fibers) from the mixture.

In some embodiments, the conditions suitable for drying comprise applying vacuum to the mixture for a time period sufficient for removing of at least 99%, at least 99.9%, at least 99.99%, at least 99.999% or more of the solvent (e.g. the liquid and water content of the wet fibers) from the mixture. In some embodiments, the conditions suitable for drying comprise conditions suitable for lyophilization of the mixture.

In some embodiments, the conditions suitable for drying comprise (i) exposing the mixture under conditions suitable for freezing the mixture, thereby obtaining a frozen mixture; and (ii) exposing the frozen mixture to vacuum for a time sufficient for substantially removing the solvent (e.g. the liquid and water content of the wet fibers). In some embodiments, the conditions suitable for drying comprise (i) providing the mixture under temperature below the freeze point of the mixture, thereby obtaining a frozen mixture; and (ii) applying sufficient vacuum to the frozen mixture for a time period sufficient for removing of at least 99%, at least 99.9%, at least 99.99%, at least 99.999% or more of the solvent (e.g. the liquid and water content of the wet fibers) from the mixture. In some embodiments, the steps (i) and (ii) are performed subsequently or simultaneously. In some embodiments, the conditions suitable for drying are so as to obtain the MaSp based fiber of the invention characterized by a BET as described herein, and optionally by a residual water content and/or by a residual amount of the solvent, as described herein.

In some embodiments, the liquid optionally comprises an aqueous solvent. In some embodiments, the liquid comprises between 50 and 100%w/w, between 50 and 70% w/w, between 70 and 80% w/w, between 80 and 100% w/w, between 80 and 90% w/w, between 90 and 95% w/w, between 95 and 99% w/w, between 99 and 100% w/w, including any range between, of an organic solvent (e.g. t-butanol); and optionally between 30 and 0.1% w/w, optionally between 30 and 20% w/w, between 20 and 0.1% w/w, between 20 and 10% w/w, between 10 and 0.1% w/w, water or an aqueous solution, including any range between.

In some embodiments, the organic solvent is capable of forming azeotrope with water. In some embodiments, the organic solvent is at a w/w ratio to the wet MaSp-based fiber, sufficient for obtaining a dry MaSp-based fiber of the invention. In some embodiments, the organic solvent is at a w/w ratio to the wet MaSp-based fiber, sufficient for forming an azeotrope with water content of the wet MaSp-based fiber. In some embodiments, the step a of the method comprises: contacting or mixing a wet MaSp-based fiber (e.g. obtained from the expression system and comprising water) with a sufficient amount of the liquid, wherein the liquid is as described herein. In some embodiments, the step a of the method comprises: contacting or mixing a wet MaSp-based fiber (e.g. obtained from the expression system and comprising water) with a liquid comprising an organic solvent to obtain a mixture; wherein a w/w ratio of the liquid to the wet MaSp-based fiber is sufficient for forming an azeotrope. In some embodiments, the step b of the method comprises providing the mixture under conditions sufficient for evaporating or removing the azeotrope. A skilled artisan will be able to adjust the liquid/fiber weight ratio, so as to result in an optimal drying or lyophilization of the MaSp-based fiber. Optimal drying conditions as well as the optimal liquid/fiber weight ratio can be assessed by calculating the porosity of the dry (e.g. lyophilized) MaSp-based fiber, as disclosed herein.

In some embodiments, the organic solvent is selected from the group consisting of ethanol, isopropyl alcohol, t-butanol, 2-butanol, n-butanol, acetonitrile, dichloromethane, benzene, ethyl acetate, DMF, DMSO, THF, TFA, toluene, hexane, heptane, methyl ethyl ketone, diethyl ketone, methyl isobutyl ketone, dioxane, and any combination thereof In some embodiments, the solvent is t-butanol. In some embodiments, the organic solvent is as described hereinabove (e.g. Class 3 solvent).

In some embodiments, conditions suitable for substantially removing the liquid from the mixture comprises centrifugation, filtration, sedimentation, lyophilization, or any combination thereof.

In another aspect of the invention, there is a method for obtaining a lyophilized major ampullate spidroin protein (MaSp)-based fiber, the method comprising: a. contacting the MaSp-based fiber with an organic solvent for a period of time; and b. separating the MaSp-based fiber from the solvent, thereby obtaining the MaSp-based fiber.

In another aspect of the invention, there is a method for purifying major ampullate spidroin protein (MaSp)-based fiber, the method comprising: a. contacting the MaSp-based fiber with an organic solvent for a period of time; and b. separating the MaSp-based fiber from the solvent, thereby purifying the MaSp-based fiber.

In some embodiments, the method comprises repeating step a. between 1 time and 5 times, 2 times and 5 times, or between 3 times and 5 times, including any range therebetween. Each possibility represents a separate embodiment of the invention.

In some embodiments, the period of time is between 10 seconds (s) and 10 minutes (min).

In some embodiments, the contacting comprises mixing, high shear mixing, overhead stirring, homogenizing, or a combination thereof.

In some embodiments, the separating comprises centrifugation, filtration, sedimentation, lyophilization, or any combination thereof.

In another aspect of the invention, there is a dried porous major ampullate spidroin protein (MaSp)-based fiber obtained by the method described hereinabove.

In another aspect of the invention, there is a lyophilized porous major ampullate spidroin protein (MaSp)-based fiber obtained by the method described hereinabove.

In some embodiments, the dried MaSp-based fiber is characterized by an improved loading capacity of an additional agent. In some embodiments, the dried MaSp-based fiber from an organic solvent is characterized by an improved loading capacity of an additional agent compared to a dried MaSp-based fiber from water. In some embodiments, the dried MaSp-based fiber from an organic solvent, is characterized by an improved loading capacity of an additional agent, compared to a control. In some embodiments, a control is a MaSp-based fiber dried from water. As used herein “loading capacity” refers to the amount of an additional agent loaded per unit weight of the dried MaSp-based fiber within the composite of the invention. Loading capacity can be calculated by the amount of the total entrapped additional agent divided by the total dried MaSp-based fiber weight within the composite of the invention.

In some embodiments, the improved loading capacity comprises a w/w ratio between the additional agent and the porous MaSp based fiber within the composite of the invention is from 100:1 to 1:10. In some embodiments, a w/w ratio between the additional agent to the MaSp-based fiber is from 100:1 to 1:10, from 100:1 to 8:1, from 100:1 to 6:1, from 100:1 to 4:1, 100:1 to 1:1, from 100:1 to 10:1, 90:1 to 1:10, from 90:1 to 8:1, from 90:1 to 6:1, from 90:1 to 4:1, 90:1 to 1:1, from 90:1 to 10:1, 50:1 to 1:10, from 50:1 to 8:1, from 50:1 to 6:1, from 50:1 to 4:1, 50:1 to 1:1, from 50:1 to 10:1, 20:1 to 1:10, from 20:1 to 8:1, from 20:1 to 6:1, from 20:1 to 4:1, 20:1 to 1:1, from 20:1 to 10:1, 10:1 to 1:10, from 10:1 to 8:1, from 8:1 to 6:1, from 6:1 to 4:1, from 4:1 to 3:1, from 3:1 to 2:1, from 2:1 to 1:1, from 1:1 to 1:2, from 1:2 to 1:3, from 1:3 to 1:5, from 1:5 to 1:10 including any range therebetween. In some embodiments, improved is as compared to a control, as described herein. Each possibility represents a separate embodiment of the invention.

In some embodiments, a w/w ratio between the additional agent and the MaSp-based fiber within the composite of the invention is at most 100:1, at most 80:1, at most 40:1, at most 20:1, at most 10:1, at most 6:1, at most 5:1, at most 4:1, at most 3:1, at most 2:1, at most 1:1 including any range therebetween. Each possibility represents a separate embodiment of the invention.

In some embodiments, the dried MaSp-based fiber is characterized by an improved retention of an additional agent. In some embodiments, the dried MaSp-based fiber from an organic solvent is characterized by an improved retention of an additional agent compared to a dried MaSp-based fiber from water.

In another aspect of the invention, there is a composite comprising the dried MaSp-based fiber and an additional agent. In some embodiments, the composite comprises the MaSp-based fiber bound to an additional agent (e.g. via physical adsorption and/or by non-covalent bonds or interactions). In some embodiments, the composition comprising the MaSp-based fiber and the additional agent is referred to herein as “the composite”. In some embodiments, the composite comprises the additional agent homogenously distributed within the matrix of the MaSp-based fibers of the invention. In some embodiments, the composite comprises the additional agent encapsulated, adsorbed or embedded within the MaSp-based fibers of the invention (e.g. within the lumen or void space, as described herein). In some embodiments, the composite of the invention is substantially homogenous or uniform (e.g. substantially devoid of aggregates, and/or phase separation).

As used herein, the term “composite” refers to a matter produced from two or more constituent materials with notably dissimilar chemical or physical properties that, when merged, create a matter with properties, unlike the individual elements.

In some embodiments, the composition and/or the composite of the invention is substantially stable. In some embodiments, a composite comprising the dried MaSp-based fiber (also used herein as “the MaSp-based fiber”, or “the MaSp-based fiber of the invention”) from an organic solvent and an additional agent has an improved stability when compared to a composite comprising the dried MaSp-based fiber from water and an additional agent. In some embodiments, the additional agent is stably encapsulated within the plurality of pores of the particle. In some embodiments, the terms “improved stability” and “stably encapsulated” refer to the ability of the composite to substantially prevent a release of the additional agent therefrom. In some embodiments, the term “improved stability” refers to the ability of the composite to substantially retain its physical properties and/or chemical composition. In some embodiments, the term “improved stability” refers to the ability of the composite to be substantially devoid of aggregation, and/or phase separation, and/or leakage of the additional agent under appropriate storage conditions, such as within a formulation (e.g. a cosmeceutical and/or pharmaceutical composition). In some embodiments, the composition and/or the composite is referred to as stable, when it is substantially devoid of phase separation for a time period disclosed herein. As used herein the term “stable” is referred to the chemical stability and/or physical stability of the composition and/or the composite of the invention.

In some embodiments, the composition and/or the composite of the invention is referred to as stable, when the concentration of the additional agent within the composition and/or the composite of the invention decreases by not more than 1%, not more than 5%, not more than 10% over 6 months at a temperature below 20° C. In some embodiments, the composition and/or the composite is referred to as stable, when the concentration of additional agent within the composition and/or the composite decreases by not more than 1%, not more than 5%, not more than 10%, including any range between, over 6 months under appropriate storage conditions, as described herein.

In some embodiments, appropriate storage conditions comprise storage temperature of between 1 and 60° C., between 1 and 10° C., between 10 and 30° C., between 30 and 40° C., between 40 and 50° C., between 50 and 60° C. including any range between. In some embodiments, appropriate storage conditions comprise ambient atmosphere. In some embodiments, appropriate storage conditions comprise storage temperature as described herein, and storage time of at least 1 month (m), at least 2 m, at least 3 m, at least 4 m, at least 5 m, at least 6 m, at least 7 m, at least 8 m, at least 10 m, at least 12 m, at least 2 years, including any range or value therebetween. In some embodiments, the term “stable” refers to a storage stability of the composition and/or composite, wherein storage stability comprises stability under appropriate storage conditions, as described herein.

In some embodiments, the dried MaSp-based fiber is characterized by a sustained release profile, compared to a control. As used herein the term “sustained release” refers to the ability of releasing an additional agent gradually and slowly, for an extended period of time, allowing for a sustained effect (e.g. as compared to a control).

In some embodiments, the encapsulated additional agent is characterized by a gradual release profile (e.g. on the application site or in a solution). In some embodiments, the particle encapsulating the additional agent substantially prevents a rapid release of the additional agent therefrom.

In some embodiments, the release rate of the additional agent from the composite comprising the dried MaSp-based fiber from an organic solvent and an additional agent is reduced by at least 10%, compared to a composite comprising the dried MaSp-based fiber from water and an additional agent. In some embodiments, the release rate of the additional agent from the composite comprising the dried MaSp-based fiber from an organic solvent and an additional agent is reduced by at least 10%, at least 20%, at least 30%, at least 50%, at least 70%, at least 90%, at least 100%, at least 150%, at least 200%, at least 250%, at least 300%, at least 400%, at least 450%, at least 500%, at least 600%, at least 700%, at least 800%, or at least 1000%, compared to a composite comprising the dried MaSp-based fiber from water and an additional agent, including any range or value therebetween. Each possibility represents a separate embodiment of the invention.

In some embodiments, the release rate of the additional agent from the composite comprising the dried MaSp-based fiber from an organic solvent and an additional agent is reduced by at least 10%, compared to a control.

In some embodiments, the control is a MaSp-based fiber lyophilized from water. As exemplified hereinbelow (FIGS. 3 and 4), the composite of the invention is characterized by an improved release profile (i.e. reduced release rate) of the additional agent compared to a control (e.g. composite comprising Masp-based fiber lyophilized from water).

In some embodiments, the release period is prolonged by at least 50%, at least 100%, at least 200%, at least 300%, at least 400%, at least 500%, at least 600%, at least 700%, at least 800%, at least 1000% compared to a control, including any range or value therebetween. Each possibility represents a separate embodiment of the invention.

In another aspect, there is a method for supplementing a subject with an additional agent, comprising administering to the subject the composition or the article of the invention; thereby supplementing the subject with the additional agent. In some embodiments, the subject is selected from a human subject or an animal subject.

### Extrudate

According to some embodiments, the present invention provides an extrudate comprising the MaSp-based fiber of the invention and/or a composition comprising thereof, and a high melt temperature polymer. In some embodiments, the MaSp-based fiber of the invention and/or extrudate comprising thereof is extrudable or moldable (e.g. being characterized by sufficient physical and/or chemical stability upon processing thereof by extrusion and/or molding). In some embodiments, molding is selected from the group consisting of: melt-extrusion, injection molding, spinning, melt-blowing and thermoforming or any combination thereof. In some embodiments, the extrudate is in a form of a composite, as described herein. In some embodiments, the extrudate is in a form of a high melt temperature polymer enriched with the MaSp-based fiber of the invention.

In some embodiments, the MaSp-based fiber and/or the extrudate comprising thereof is stable upon exposing thereof to conditions suitable for molding or extrusion. In some embodiments, conditions suitable for molding comprise exposing the MaSp-based fiber and/or the extrudate to a temperature about the softening point. In some embodiments, conditions suitable for molding comprise exposing the MaSp-based fiber and/or the extrudate to a temperature about the Tm (of the MaSp-based fiber and/or of the extrudate). In some embodiments, conditions suitable for molding comprise thermal exposure in a range between 100 and 300° C., thereby melting or softening the extrudate. In some embodiments, conditions suitable for molding or extrusion comprise providing the extrudate under conditions suitable for melting or softening thereof.

In some embodiments, a weight per weight (w/w) ratio of the MaSp-based fiber to the high melt temperature polymer is between 0.01:1 and 1:1, 0.02:1 and 1:1, 0.05:1 and 1:1, 0.09:1 and 1:1, 0.1:1 and 1:1, 0.5:1 and 1:1, or between 0.9:1 and 1:1, including any range therebetween. Each possibility represents a separate embodiment of the invention.

In some embodiments, the extrudate comprises 0.01% to 50% (w/w), 0.05% to 50% (w/w), 0.09% to 50% (w/w), 0.1% to 50% (w/w), 0.5% to 50% (w/w), 0.9% to 50% (w/w), 1% to 50% (w/w), 5% to 50% (w/w), 10% to 50% (w/w), 15% to 50% (w/w), 20% to 50% (w/w), 30% to 50% (w/w), 0.01% to 20% (w/w), 0.05% to 20% (w/w), 0.09% to 20% (w/w), 0.1% to 20% (w/w), 0.5% to 20% (w/w), 0.9% to 20% (w/w), 1% to 20% (w/w), 5% to 20% (w/w), 0.01% to 10% (w/w), 0.05% to 10% (w/w), 0.09% to 10% (w/w), 0.1% to 10% (w/w), 0.5% to 10% (w/w), 0.9% to 10% (w/w), or 1% to 10% (w/w), of the MaSp-based fiber, including any range therebetween. Each possibility represents a separate embodiment of the invention.

In some embodiments, the high melt temperature polymer is bound to the MaSp-based fiber via a non-covalent bond, a physical interaction, or both.

In some embodiments, the high melt temperature polymer is characterized by a melting temperature (Tm) between 200° C. and 400° C., 210° C. and 400° C., 220° C. and 400° C., 250° C. and 400° C., 270° C. and 400° C., 280° C. and 400° C., 200° C. and 390° C., 210° C. and 390° C., 220° C. and 390° C., 250° C. and 390° C., 270° C. and 390° C., 280° C. and 390° C., 200° C. and 380° C., 210° C. and 380° C., 220° C. and 380° C., 250° C. and 380° C., 270° C. and 380° C., 280° C. and 380° C., 200° C. and 350° C., 210° C. and 350° C., 220° C. and 350° C., 250° C. and 350° C., 270° C. and 350° C., 280° C. and 350° C., 200° C. and 310° C., 210° C. and 310° C., 220° C. and 310° C., 250° C. and 310° C., 270° C. and 310° C., 280° C. and 310° C., 200° C. and 300° C., 210° C. and 300° C., 220° C. and 300° C., 250° C. and 300° C., 270° C. and 300° C., or between 280° C. and 380° C., including any range therebetween. Each possibility represents a separate embodiment of the invention.

As used herein, the term “high melt temperature polymer” refers to a polymer characterized by a continuous use temperature (CUT) or relative thermal index (RTI) of greater than 150° C. These temperatures are considered to be the maximum useful service temperature for materials where a critical property will not be unacceptably compromised through thermal degradation. The maximum continuous use temperature is the maximum acceptable temperature above which mechanical properties (tensile strength, impact strength) or electrical properties (dielectric strength, linked to insulation properties) of a material are significantly degrading, over the reasonable life time of the tested product.

In some embodiments, the high melt temperature polymer is a thermoplastic polymer. As used herein “thermoplastic” refers to a material which becomes softer when heated and hard when cooled. Thermoplastic materials can be cooled and heated several times without any change in their chemical or mechanical properties.

In some embodiments, high temperature thermoplastics are also referred to as “high performance plastics” or “high performance polymers”. As used herein, the term high performance polymer” refers to a group of polymer materials that are known to retain its desirable mechanical, thermal, and chemical properties when subjected to harsh environmental conditions such as high temperature, high pressure, and corrosive chemicals.

In some embodiments, the high melt temperature polymer is selected from the group consisting of poly(vinyl chloride) (PVC), poly(6-aminocaproic acid), poly(caprolactam), poly(hexamethylene sebacamide) (Nylon 6,10), poly(vinyl alcohol), poly(decamethylene adipamide) (Nylon 10,6), poly(hexamethylene suberamide) (Nylon 6,8), poly(styrene) (PS), poly(4-methylpentene) (PMP), poly(ethylene terephthalate) (PET), poly(hexamethylene adipamide) (Nylon 6,6), poly(acrylonitrile) (PAN), poly(tetrafluoroethylene) (PTFE), polyarylate, ethylene-vinyl acetate (EVA), polybutylene terephthalate (PBT), poly(1,4-cyclohexanedimethylene terephthalate) (PCT), polyetheretherketone (PEEK), and any copolymer and combination thereof.

In some embodiments, the MaSp-based fiber is characterized by a thermal stability at a temperature between 200° C. and 300° C. up to 5 hours. In some embodiments, the extrudate is characterized by a thermal stability at a temperature between 200° C. and 300° C., 210° C. and 300° C., 220° C. and 300° C., 250° C. and 300° C., 270° C. and 300° C., or between 280° C. and 380° C., including any range therebetween, up to 5 hours. Each possibility represents a separate embodiment of the invention. In some embodiments, the extrudate is characterized by a thermal stability at a temperature between 200° C. and 300° C., up to 5 hours, up to 2 hours, up to 1 hour, up to 40 minutes, up to 20 minutes, or up to 10 minutes, including any range therebetween. Each possibility represents a separate embodiment of the invention.

In some embodiments, the extrudate is characterized by a thermal stability at a temperature between 200° C. and 300° C., for a period of time ranging from 1 minute to 5 hours, 2 minutes to 5 hours, 5 minutes to 5 hours, 10 minutes to 5 hours, 20 minutes to 5 hours, 30 minutes to 5 hours, 1 minute to 2 hours, 2 minutes to 2 hours, 5 minutes to 2 hours, 10 minutes to 2 hours, 20 minutes to 2 hours, 30 minutes to 2 hours, 1 minute to 1 hour, 2 minutes to 1 hour, 5 minutes to 1 hour, 10 minutes to 1 hour, 20 minutes to 1 hour, 30 minutes to 1 hour, 1 minute to 40 minutes, 2 minutes to 40 minutes, 5 minutes to 40 minutes, 10 minutes to 40 minutes, 20 minutes to 40 minutes, 1 minute to 20 minutes, 2 minutes to 20 minutes, 5 minutes to 20 minutes, or 10 minutes to 20 minutes, including any range therebetween. Each possibility represents a separate embodiment of the invention.

In some embodiments, the MaSp-based fiber is characterized by a thermal stability at a temperature between 200° C. and 300° C., 210° C. and 300° C., 220° C. and 300° C., 250° C. and 300° C., 270° C. and 300° C., or between 280° C. and 380° C., including any range therebetween, up to 5 hours. Each possibility represents a separate embodiment of the invention.

In some embodiments, the MaSp-based fiber is characterized by a thermal stability at a temperature between 200° C. and 300° C., up to 5 hours, up to 2 hours, up to 1 hour, up to 40 minutes, up to 20 minutes, or up to 10 minutes, including any range therebetween. Each possibility represents a separate embodiment of the invention.

In some embodiments, the MaSp-based fiber is characterized by a thermal stability at a temperature between 200° C. and 300° C., for a period of time ranging from 1 minute to 5 hours, 2 minutes to 5 hours, 5 minutes to 5 hours, 10 minutes to 5 hours, 20 minutes to 5 hours, 30 minutes to 5 hours, 1 minute to 2 hours, 2 minutes to 2 hours, 5 minutes to 2 hours, 10 minutes to 2 hours, 20 minutes to 2 hours, 30 minutes to 2 hours, 1 minute to 1 hour, 2 minutes to 1 hour, 5 minutes to 1 hour, 10 minutes to 1 hour, 20 minutes to 1 hour, 30 minutes to 1 hour, 1 minute to 40 minutes, 2 minutes to 40 minutes, 5 minutes to 40 minutes, 10 minutes to 40 minutes, 20 minutes to 40 minutes, 1 minute to 20 minutes, 2 minutes to 20 minutes, 5 minutes to 20 minutes, or 10 minutes to 20 minutes, including any range therebetween. Each possibility represents a separate embodiment of the invention.

In some embodiments, the MaSp-based fiber is characterized by a compressive strength in the range of 10000 psi to 24000 psi, 12000 psi to 24000 psi, 15000 psi to 24000 psi, 18000 psi to 24000 psi, 20000 psi to 24000 psi,10000 psi to 21000 psi, 12000 psi to 21000 psi, 15000 psi to 21000 psi, 18000 psi to 21000 psi, 20000 psi to 21000 psi, 10000 psi to 20000 psi, 12000 psi to 20000 psi, 15000 psi to 20000 psi, 18000 psi to 20000 psi, 10000 psi to 17000 psi, 12000 psi to 17000 psi, or 15000 psi to 17000 psi, including any range therebetween. Each possibility represents a separate embodiment of the invention.

In some embodiments, the MaSp-based fiber is characterized by a flexural strength in the range of 12000 psi to 25000 psi, 12000 psi to 21000 psi, 15000 psi to 21000 psi, 18000 psi to 21000 psi, 20000 psi to 21000 psi, 12000 psi to 20000 psi, 15000 psi to 20000 psi, 18000 psi to 20000 psi, 12000 psi to 17000 psi, or 15000 psi to 17000 psi, including any range therebetween. Each possibility represents a separate embodiment of the invention.

The term “extrudate” refers to a product in which the composition is heated and/or compressed to a molten (or softened) state and subsequently extruded. As used herein, the term “extrudable composition” refers to the ability of a composition to be extruded. “Extrudate” and “extrudable composition” are used herein to refer not only to a composition that contains polymers with thermoplastic properties, but also those polymers that are readily extrudable by known techniques or otherwise behave similar to thermoplastic polymers with respect to extrusion processes.

In some embodiments, the extrudate is characterized by an improved thermal stability as compared to the thermal stability of the high melting temperature polymer free of the MaSp-based fiber.

In some embodiments, the extrudate is characterized by at least one improved mechanical property as compared to the property for the high melting temperature polymer free of the MaSp-based fiber, wherein the property is selected from the group consisting of: Young's modulus, storage modulus, loss modulus, tensile strength, fracture strain, yield point, toughness, work to failure, impact strength, tear strength, flexural modulus, flexural strain and stress at a specific percentage elongation, and abrasion.

According to some embodiments, the present invention provides an article comprising an extrudate as described hereinabove.

In some embodiments, the article is in the form of reinforced plastics, a bottle, a container, a package, a cable, a tube, a film, a rope, a thread, or a textile.

In some embodiments, the article is characterized by at least one improved mechanical property as compared to the property for the article free of the composition, wherein the property is selected from the group consisting of: Young's modulus, tensile strength, fracture strain, yield point, toughness, work to failure, impact strength, tear strength, flexural modulus, flexural strain and stress at a specific percentage elongation, abrasion, UV-resistance and gas permeability.

In some embodiments, the present invention provides an abrasion resistant extrudate. In some embodiments, the present invention provides an extrudate with improved abrasion resistance. As used herein, the term “abrasion resistance” refers to the ability of a material to stop the displacement when exposed to a relative movement of the hard particles or projections. Abrasion resistance can be measured through a variety of tests known in the art, such as for example, burned off (Taber) wear test, Gardner scrubber (Gardner scrubber) test, a sand-fall (falling sand) tests.

In some embodiments, one or more properties selected from thermal stability, Young's modulus, tensile strength, yield point, abrasion resistance, and stress at elongation, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, one or more properties selected from thermal stability, Young's modulus, tensile strength, yield point, abrasion resistance, and stress at elongation, is enhanced by e.g., at least 100%, at least 150%, at least 250%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least 850%, at least 900%, at least 1000%, at least 1500%, at least 2000%, at least 2500%, or at least 3000%. Each possibility represents a separate embodiment of the invention.

In some embodiments, the composition is characterized by a Young's modulus in the range of 50 MPa to 170 MPa, 52 MPa to 170 MPa, 60 MPa to 170 MPa, 68 MPa to 170 MPa, 90 MPa to 170 MPa, 100 MPa to 170 MPa, 101 MPa to 170 MPa, 105 MPa to 170 MPa, 101 MPa to 160 MPa, or 105 MPa to 160 MPa, including any range therebetween. Each possibility represents a separate embodiment of the invention.

In some embodiments, at least two properties selected from thermal stability, Young's modulus, tensile strength, yield point, abrasion resistance, and stress at elongation, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, at least three properties selected from thermal stability, Young's modulus, tensile strength, yield point, abrasion resistance, and stress at elongation, are enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, the thermal stability is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, the Young's modulus is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

In some embodiments, the tensile strength is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least 50%. Each possibility represents a separate embodiment of the invention.

In some embodiments, the yield point is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least 50%. Each possibility represents a separate embodiment of the invention.

In some embodiments, the abrasion resistance is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least 50%. Each possibility represents a separate embodiment of the invention.

In some embodiments, the extrudate is characterized by a structural strength, wherein more than 20% of the structural strength results from the incorporated MaSp-based fiber. In some embodiments, the composite is characterized by a structural strength, wherein more than 30% of the structural strength results from the incorporated MaSp-based fiber.

In some embodiments, the extrudate is characterized by a structural strength, wherein more than 1% of the tensile strength results from the incorporated MaSp-based fiber. In some embodiments, the extrudate is characterized by a structural strength, wherein more than 5% of the tensile strength results from the incorporated MaSp-based fiber. In some embodiments, the extrudate is characterized by a structural strength, wherein more than 10% of the tensile strength results from the incorporated MaSp-based fiber. In some embodiments, the extrudate is characterized by a structural strength, wherein more than 20% of the tensile strength results from the incorporated MaSp-based fiber. In some embodiments, the extrudate is characterized by a tensile strength, wherein more than 30% of the structural strength results from the incorporated MaSp-based fiber.

### MaSp-based Fibers

The terms “major ampullate spidroin protein” and “spidroin protein” are used interchangeably throughout the description and encompass all known major ampullate spidroin proteins, typically abbreviated “MaSp”, or “ADF” in the case of Araneus diadematus. These major ampullate spidroin proteins are generally of two types, 1 and 2. These terms furthermore include non-natural proteins, as disclosed herein, with a high degree of identity and/or similarity to at least the repetitive region of the known major ampullate spidroin proteins. Additional suitable spider silk proteins include MaSp2, MiSp, MiSp2, AcSp, FLYS, FLAS, and flagelliform.

As used herein, the term “repetitive region”, “repetitive sequence” or “repeat” refer to a recombinant protein sequence derived from repeat units which naturally occur multiple times in spider silk amino acid sequences (e.g., in the MaSp-1 protein). One skilled in the art will appreciate that the primary structure of the spider silk proteins is considered to consist mostly of a series of small variations of a unit repeat. The unit repeats in the naturally occurring proteins are often distinct from each other. That is, there is little or no exact duplication of the unit repeats along the length of the protein. In some embodiments, the synthetic spider silks of the invention are made wherein the primary structure of the protein comprises a number of exact repetitions of a single unit repeat. In additional embodiments, synthetic spider silks of the invention comprise a number of repetitions of one unit repeat together with a number of repetitions of a second unit repeat. Such a structure would be similar to a typical block copolymer. Unit repeats of several different sequences may also be combined to provide a synthetic spider silk protein having properties suited to a particular application. The term “direct repeat” as used herein is a repeat in tandem (head-to-tail arrangement) with a similar repeat. In another embodiment, the repeat used to form the synthetic spider silk of the invention is a direct repeat. In some embodiments, the repeat is not found in nature (i.e., is not a naturally occurring amino acid sequences).

An exemplary sequence comprising repetitive sequences is ADF-4: AAAAAAASGSGGYGPENQGPSGPVAYGPGGPVSSAAAAAAAGSGPGGYGPENQ GPSGPGGYGPGGSGSSAAAAAAAASGPGGYGPGSQGPSGPGGSGGYGPGSQGPS GPGASSAAAAAAAASGPGGYGPGSQGPSGPGAYGPGGPGSSAAASGPGGYGPGS QGPSGPGGSGGYGPGSQGPSGPGGPGASAAAAAAAAASGPGGYGPGSQGPSGPG AYGPGGPGSSAAASGPGGYGPGSQGPSGPGAYGPGGPGSSAAAAAAAGSGPGGY GPGNQGPSGPGGYGPGGPGSSAAAAAAASGPGGYGPGSQGPSGPGVYGPGGPGS SAAAAAAAGSGPGGYGPGNQGPSGPGGYGPGGSGSSAAAAAAAASGPGGYGPG SQGPSGPGGSGGYGPGSQGPSGPGASSAAAAAAAASGPGGYGPGSQGPSGPGAY GPGGPGSSAAASGPGGYGPGSQGPSGPGAYGPGGPGSSAAAAAAASGPGGYGPG SQGPSGPGGSRGYGPGSQGPGGPGASAAAAAAAAASGPGGYGPGSQGPSGPGYQ GPSGPGAYGPSPSASAS (SEQ ID NO: 1). In some embodiments, the synthetic repetitive sequence of the invention is based on (e.g., has a high percentage identity, as defined hereinbelow) one or more repetitive sequences derived from ADF-4 (SEQ ID NO: 1). As used herein, the term “based on” refers to a sequence having a high percentage of homology to a repetitive sequence.

In some embodiments, each repetitive sequence comprises up to 60 amino acids, up to 55 amino acids, up to 50 amino acids, up to 49 amino acids, up to 48 amino acids, up to 47 amino acids, up to 46 amino acids, up to 45 amino acids, up to 44 amino acids, up to 43 amino acids, up to 42 amino acids, up to 41 amino acids, up to 40 amino acids, up to 39 amino acids, up to 38 amino acids, up to 37 amino acids, up to 36 amino acids or up to 35 amino acids, wherein possibility represents a separate embodiment of the present invention. In some embodiments, each repetitive sequence comprises 5 to 60 amino acids, 10 to 55 amino acids, 15 to 50 amino acids, 20 to 45 amino acids, 25 to 40 amino acids, acids, 25 to 39 amino acids or 28 to 36 amino acids, wherein possibility represents a separate embodiment of the present invention. In some embodiments, each repetitive sequence comprises 30 to 40 amino acids, 31 to 39 amino acids, 32 to 38 amino acids, 33 to 37 amino acids, 34 to 36 amino acids, wherein each possibility represents a separate embodiment of the present invention. In an additional embodiment, each repetitive sequence comprises 35 amino acids.

In some embodiments, the repetitive region comprises, independently, an amino acid sequence as set forth in Formula 1 (X₁)_{Z}X₂GPGGYGPX₃X₄X₅GPX₆GX₇GGX₈GPGGPGX₉X₁₀; wherein X₁ is, independently, at each instance A or G.

In some embodiments, at least 50% of (X₁)z is A, Z is an integer between 5 to 30; X₂ is S or G; X₃ is G or E; X₄ is G, S or N; X₅ is Q or Y; X₆ is G or S; X₇ is P or R; X₈ is Y or Q; X₉ is G or S; and X₁₀ is S or G.

In another embodiment, the repetitive region of a MaSP1 protein comprises the amino acid sequence as set forth in SEQ ID NO: 2 (SGPGGYGPGSQGPSGPGGYGPGGPGSS). In another embodiment, the repetitive region of a MaSP1 protein comprises the amino acid sequence as set forth in SEQ ID NO: 3 (AAAAAAAASGPGGYGPGSQGPSGPGGYGPGGPGSS).

In another embodiment, there is provided a homolog of the repetitive region of a MaSP1 protein sharing at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% homology with SEQ ID NO: 1. Each possibility represents a separate embodiment of the invention.

In another embodiment, the homolog shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% homology with SEQ ID NO: 2. Each possibility represents a separate embodiment of the invention.

In another embodiment, the repetitive region of a MaSP1 protein has the amino acid sequence as set forth in SEQ ID NO: 1.

In another embodiment, the MaSP1 protein comprises a single N-terminal region selected from the group consisting of: SEQ ID NO: 4 (MSYYHHHHHHDYDIPTTENLYFQGAMDPEFKGLRRRAQLV); SEQ ID NO: 5 (MSYYHEIHHHHDYDIPTTENLYFQGAMDPEFKGLRRRAQLVRPLSNLDNAP); SEQ ID NO: 6 (MSYYHEIHHHHDYDIPTTENLYFQGAMDPEFKGLRRRAQLVDPPGCRNSARAGS S), or any functional homolog, variant, derivative, or fragment thereof. In another embodiment, the homolog of the C-terminal region shares at least 70% homology with any one of SEQ ID NOs: 4-6.

In another embodiment, the MaSP1 protein further comprises a single C-terminal region selected from the group consisting of: SEQ ID NO: 7 (VAASRLSSPAASSRVSSAVSSLVSSGPTNGAAVSGALNSLVSQISASNPGLSGCD ALVQALLELVSALVAILSSASIGQVNVSSVSQSTQMISQALS); and SEQ ID NO: 8 (GPSGPGAYGPSPSASASVAASRLSSPAASSRVSSAVSSLVSSGPTNGAAVSGALN SLVSQISASNPGLSGCDALVQALLELVSALVAILSSASIGQVNVSSVSQSTQMISQA LS), or any functional homolog, variant, derivative, fragment or mutant thereof. In another embodiment, the homolog of the N-terminal region shares at least 70% homology with SEQ ID NO: 7-8.

In some embodiments, the MaSp-based fibers comprising a mixture of proteins, as disclosed under WO2017025964, which is incorporated herein by reference in its entirety.

In some embodiments, the MaSP1 protein further comprises at least one tag sequence. Non-limiting examples of tags which may be used in the present invention include a His tag, a HA tag, a T7 tag, and the like. The skilled person is well aware of alternative suitable tags or other fusion partners.

“Amino acid” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Amino acid sequence” or “peptide sequence” is the order in which amino acid residues, connected by peptide bonds, lie in the chain in peptides and proteins. The sequence is generally reported from the N-terminal end containing free amino group to the C-terminal end containing free carboxyl group Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, sulfhydryl bond formation, cleavage and the likes.

As used herein, “isolated” or “substantially purified”, in the context of synthetic spider silk amino-acid sequences or nucleic acid molecules encoding the same, as exemplified by the invention, means the amino-acid sequences or polynucleotides have been removed from their natural milieu or have been altered from their natural state. As such “isolated” does not necessarily reflect the extent to which the amino-acid sequences or nucleic acid molecules have been purified. However, it will be understood that such molecules that have been purified to some degree are “isolated”. If the molecules do not exist in a natural milieu, i.e. it does not exist in nature, the molecule is “isolated” regardless of where it is present. By way of example, amino-acid sequences or polynucleotides that do not naturally exist in humans are “isolated” even when they are present in humans.

The term “isolated” or “substantially purified”, when applied to an amino acid sequence or nucleic acid, denotes that the amino acid sequence or nucleic acid is essentially free of other cellular components with which they are associated in the natural state. It may be in a homogeneous state, or alternatively in either a dry or aqueous solution. Purity and homogeneity are typically determined using analytical chemistry techniques such as polyacrylamide gel electrophoresis or high-performance liquid chromatography. An amino acid sequence or nucleic acid which is the predominant species present in a preparation is substantially purified.

In some embodiments, the repeats are of a homolog, variant, derivative of a repetitive region of a MaSp1 protein or fragment thereof. In some embodiments, the repeats are of a homolog, variant, derivative of a repetitive region of an ADF-4 protein or fragment thereof.

As used herein, the term “functional” as in “functional homolog, variant, derivative or fragment”, refers to an amino acid sequence which possesses biological function or activity that is identified through a defined functional assay. More specifically, the defined functional assay is the formation of self-assembling fibers in cells expressing the functional homolog, variant, derivative or fragment.

An amino acid sequence or a nucleic acid sequence is the to be a homolog of a corresponding amino acid sequence or a nucleic acid, when the homology is determined to be at least 50%, at least 55%, at least 60%, at least 65%, at least 70%, at least 75%, at least 80%, at least 85%, at least 90%, at least 92%, at least 94%, at least 96%, at least 98% or at least 99%.

The terms “identical”, “substantial identity”, “substantial homology” or percent “identity”, in the context of two or more amino acids or nucleic acids sequences, refer to two or more sequences or subsequences that are the same or have a specified percentage of amino acid residues or nucleotides that are the same (i.e., about 60% identity, or at least 65%, at least 70%, at least 75%, at least 80%, at least 85%, at least 90%, at least 91%, at least 92%, at least 93%, at least 94%, at least 95%, at least 96%, at least 97%, at least 98%, or 99% identity over a specified region (e.g., amino acid sequence SEQ ID NO: 2 or 3), when compared and aligned for maximum correspondence over a comparison window or designated region) as measured using a BLAST or BLAST 2.0 sequence comparison algorithms with default parameters described below, or by manual alignment and visual inspection. Such sequences are then to be “substantially identical”. This definition also refers to, or may be applied to, the compliment of a test sequence. The definition also includes sequences that have deletions and/or additions, as well as those that have substitutions. The preferred algorithms can account for gaps and the like.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

It should be appreciated that the invention further encompasses amino acid sequence comprising n repeats of a variant of any one of SEQ ID NO: 1, 2, or 3. As used herein, the term “variant” or “substantially similar” comprises sequences of amino acids or nucleotides different from the specifically identified sequences, in which one or more (e.g., 1, 2, 3, 4, 5, 6, 7, 8, 9, 10, 20 or 25) amino acid residues or nucleotides are deleted, substituted or added. The variants may be allelic variants occurring naturally or variants of non-natural origin. The variant or substantially similar sequences refer to fragments of amino acid sequences or nucleic acids that may be characterized by the percentage of the identity of their amino acid or nucleotide sequences with the amino acid or nucleotide sequences described herein, as determined by common algorithms used in the state-of-the-art. The preferred fragments of amino acids or nucleic acids are those having a sequence of amino acids or nucleotides with at least around 40 or 45% of sequence identity, preferentially around 50% or 55% of sequence identity, more preferentially around 60% or 65% of sequence identity, more preferentially around 70% or 75% of sequence identity, more preferentially around 80% or 85% of sequence identity, yet more preferentially around 90%, 91%, 92%, 93%, 94%, 95%, 96%, 97%, 98% or 99% of sequence identity when compared to the sequence of reference. Each possibility represents a separate embodiment of the invention.

In one embodiment, the MaSp-based polymer is a fiber.

In one embodiment, the MaSp-based polymer is composed of monomers. In one embodiment, a plurality of monomers are arranged in a nanofibril. In one embodiment, a plurality of nanofibrils are arranged in a fiber or make-up a fiber. In one embodiment, a monomer or a nanofibril within the MaSp-based polymer or a fiber has a diameter of 4 to 16 nm. In one embodiment, a monomer or a nanofibril within the MaSp-based polymer or a fiber has a diameter of 6 to 14 nm. In one embodiment, a monomer or a nanofibril within the MaSp-based polymer or a fiber has a diameter of 8 to 12 nm. In one embodiment, a fiber or the MaSp-based polymer has a diameter of 70 to 450 nm. In one embodiment, a fiber or the MaSp-based polymer of proteins has a diameter of 80 to 350 nm. In one embodiment, a fiber the MaSp-based polymer has a diameter of 80 to 300 nm. In one embodiment, a fiber or the MaSp-based polymer has a diameter of 150 to 250 nm. In one embodiment, a fiber or the MaSp-based polymer is arranged as a coil. In one embodiment, a single fiber or one the MaSp-based polymer is arranged as a coil. In one embodiment, a coil has a diameter of 5 to 800 micrometers. In one embodiment, a coil has a diameter of 5 to 500 micrometers. In one embodiment, a coil has a diameter of 5 to 30 micrometers. In one embodiment, a coil has a diameter of 5 to 20 micrometers. In one embodiment, a fiber or the MaSp-based polymer has a length of 5 to 800 micrometers. In one embodiment, a fiber or the MaSp-based polymer has a length of 30 to 300 micrometers.

In one embodiment, a fiber or the MaSp-based polymer is branched. In one embodiment, a fiber or the MaSp-based polymer comprises 1 to 10 branches. In one embodiment, a fiber or the MaSp-based polymer is free of carbohydrates. In one embodiment, a fiber or the MaSp-based polymer is non-glycosylated. In one embodiment, a fiber or the MaSp-based polymer is free of fat or fatty acids. In one embodiment, a fiber or the MaSp-based polymer is free of phosphorus. In one embodiment, a fiber or the MaSp-based polymer is free of an additional non-MaSp-based protein. In one embodiment, a fiber or the MaSp-based polymer is free of an additional polymer (e.g. a synthetic polymer, a non-MaSp-based peptide, non-MaSp-based protein). In one embodiment, a fiber or the MaSp-based polymer is substantially free of an additional polymer. In one embodiment, “free of” is “devoid of” or essentially “devoid of”.

In one embodiment, the aspect ratio of length to diameter of a fiber the MaSp-based polymer is at least 1:10. In one embodiment, the aspect ratio of length to diameter of a fiber or the MaSp-based polymer is at least 1:10 to 1:1500. In one embodiment, the aspect ratio of length to diameter of a fiber or the MaSp-based polymer is at least 1:50 to 1:1000. In one embodiment, the aspect ratio of length to diameter of a fiber or the MaSp-based polymer is at least 1:100 to 1:1200. In one embodiment, the aspect ratio of length to diameter of a fiber or the MaSp-based polymer is at least 1:100 to 1:1000. In one embodiment, the aspect ratio of length to diameter of a fiber or the MaSp-based polymer is at least 1:500 to 1:1000.

The terms derivatives and functional derivatives as used herein mean the amino acid sequence of the invention with any insertions, deletions, substitutions and modifications.

It should be appreciated that by the term “insertions”, as used herein it is meant any addition of amino acid residues to the sequence of the invention, of between 1 to 50 amino acid residues, specifically, between 20 to 1 amino acid residues, and more specifically, between 1 to 10 amino acid residues. Most specifically, 1, 2, 3, 4, 5, 6, 7, 8, 9 and 10 amino acid residues. Further, the amino acid sequence of the invention may be extended at the N-terminus and/or C-terminus thereof with various identical or different amino acid residues.

Amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

In another embodiment, the repeat sequence of the invention has 17 or fewer, 16 or fewer, 15 or fewer, 14 or fewer, 13 or fewer, 12 or fewer, 11 or fewer, 10 or fewer, 9 or fewer, 8 or fewer, or 7 or fewer amino acid substitutions to the sequence of any one of SEQ ID NO: 2 or 3. In one embodiment, the repeat sequence of the invention has at least 2, at least 3, at least 4, at least 5, at least 6, at least 7, at least 8, at least 9, at least 10, at least 11, at least 12, or at least 13 amino acid substitutions to the sequence of any one of SEQ ID NO: 1, 2 or 3.

With respect to amino acid sequences, one of skill will recognize that individual substitutions, deletions or additions to an amino acid, nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Conservative nucleic acid substitutions are nucleic acid substitutions resulting in conservative amino acid substitutions as defined above.

Variants of the amino acid sequences of the invention may have at least 80% sequence similarity, at least 85% sequence similarity, 90% sequence similarity, or at least 95%, 96%, 97%, 98%, or 99% sequence similarity at the amino acid level, with a repeating unit denoted by one of SEQ ID NO: 2 or 3.

The amino acid sequence of the invention may comprise 2-70 repeats of SEQ ID NO. 1 or SEQ ID NO. 3 or of any fragment thereof. A “fragment” constitutes a fraction of the amino acid or DNA sequence of a particular region. A fragment of the peptide sequence is at least one amino acid shorter than the particular region, and a fragment of a DNA sequence is at least one base-pair shorter than the particular region. The fragment may be truncated at the C-terminal or N-terminal sides, or both. An amino acid fragment may comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 24, at least 26, at least 27, at least 28, at least 29, at least 30, at least 31, at least 32, at least 33 or at least 34 amino acids of SEQ ID NO: 1 or 3. Each possibility represents a separate embodiment of the invention.

Mutants of the amino acid sequences of the invention are characterized in the exchange of one (point mutant) or more, about up to 10, of its amino acids against one or more of another amino acid. They are the consequence of the corresponding mutations at the DNA level leading to different codons.

Still further, the invention concerns derivatives of the amino acid sequence of the invention. Derivatives of the amino acid sequences of the invention are, for example, where functional groups, such as amino, hydroxyl, mercapto or carboxyl groups, are derivatised, e.g. glycosylated, acylated, amidated or esterified, respectively. In glycosylated derivatives an oligosaccharide is usually linked to asparagine, serine, threonine and/or lysine. Acylated derivatives are especially acylated by a naturally occurring organic or inorganic acid, e.g. acetic acid, phosphoric acid or sulphuric acid, which usually takes place at the N-terminal amino group, or at hydroxy groups, especially of tyrosine or serine, respectively. Esters are those of naturally occurring alcohols, e.g. methanol or ethanol. Further derivatives are salts, especially pharmaceutically acceptable salts, for example metal salts, such as alkali metal and alkaline earth metal salts, e.g. sodium, potassium, magnesium, calcium or zinc salts, or ammonium salts formed with ammonia or a suitable organic amine, such as a lower alkylamine, e.g. triethylamine, hydroxy-lower alkylamine, e.g. 2-hydroxyethylamine, and the like.

In some embodiments, the silk protein of the invention is devoid of post translational modifications.

In some embodiments, the silk protein of the invention is biodegradable. This characteristic may be of importance, for example, in the field of medicine, whenever the silk proteins are intended for an in vivo use, in which biological degradation is desired. This characteristic may in particular find application in suture materials and wound closure and coverage systems.

According to some aspects, the MaSp-based fiber of the invention is manufactured using an expression vector comprising a suitable nucleic acid sequence, wherein the nucleic acid sequence is under expression control of an operably linked promoter and, optionally, regulatory sequences. Exemplary expression systems are known in the art, such as an expression system disclosed in PCT/IL2020/050752.

In some embodiments, the MaSp-based protein results in a self-assembled forming a defined structure. In some embodiments, the MaSp-based protein is in the form of a network. In some embodiments, the MaSp-based protein is in the form of a complex. In some embodiments, the MaSp-based protein induces a defined secondary structure, e.g., a beta turn, gamma turn, beta sheet, alpha helix conformation, and the like.

According to some aspects, the MaSp-based protein and/or the MaSp-based polymer which are used herein interchangeably, are in the form of a fiber, thus it should be apparent, that in some aspects of the invention the terms MaSp-based polymer and MaSp-based fiber are used herein interchangeably. A “fiber” as used herein, is meant a fine cord of fibrous material composed of two or more filaments twisted together. By “filament” is meant a slender, elongated, threadlike object or structure of indefinite length, ranging from microscopic length to lengths of a mile or greater. Specifically, the synthetic spider silk filament is microscopic, and is proteinaceous. By “biofilament” is meant a filament created from a protein, including recombinantly produced spider silk protein. In some embodiments, the term “fiber” does not encompass unstructured aggregates or precipitates.

In some embodiments, the fiber of the proteins is characterized by size of at least one dimension thereof (e.g., diameter, length). For example, and without limitation, the diameter of the fiber is between 10 nm-1 μm, 20-100 nm, or 10-50 nm.

In some embodiments, the fiber is composed of nanofibrils. In some embodiments, the nanofibrils have a diameter of e.g., 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 40 nm, about 42 nm, about 44 nm, about 46 nm, about 48 nm, or about 50 nm, including any value or range therebetween. Each possibility represents a separate embodiment of the invention. In one embodiment, the nanofibrils have a diameter of 3-7 nm. In one embodiment, the nanofibrils have a diameter of 4-6 nm.

In some embodiments, the length of the disclosed fiber is between 1-200 μm, 10-100 μm, 100 to 500 μm or 200-500 μm.

In some embodiments of any one of the embodiments described herein, the disclosed fiber (e.g. particle) is characterized by a porous structure. In some embodiments, the porous structure is characterized by a porosity of at least 30% (e.g., from 30 to 99%). In some embodiments, the porous structure is characterized by a porosity of at least 50% (e.g., from 50 to 99%). In some embodiments, the porous structure is characterized by a porosity of at least 60% (e.g., from 60 to 99%). In some embodiments, the porous structure is characterized by a porosity of at least 70% (e.g., from 70 to 99%). In some embodiments, the porous structure is characterized by a porosity of at least 80% (e.g., from 80 to 99%). In some embodiments, the porous structure is characterized by a porosity of at least 90% (e.g., from 90 to 99%). In some embodiments, the porous structure is characterized by a porosity of about 90%.

Herein, the term “porosity” refers to a percentage of the volume of a substance (e.g., a “sponge-like” material) which consists of voids. In another embodiment, porosity is measured according to voids or lumens within the surface area divided to the entire surface area (porous and non-porous).

In some embodiments, the porous structure of the disclosed fibers allows absorbing water efficiently on the fiber surface. That is, and without being bound by any particular theory, this surprising discovery can be explained in view of the disclosed fiber structure and its porosity which is in sharp distinction from native spider silk found in nature.

In some embodiments of any one of the embodiments described herein, the disclosed fiber is characterized by a mean diameter is nanosized.

In some embodiments, the disclosed fiber is characterized by a mean diameter is in a range of from 1 to 50 nm. In some such embodiments, the mean diameter is in a range of from 3 to 50 nm. In some such embodiments, the mean diameter is in a range of from 5 to 50 nm. In some such embodiments, the mean diameter is in a range of from 1 to 40 nm. In some such embodiments, the mean diameter is in a range of from 1 to 30 nm. In some such embodiments, the mean diameter is in a range of from 5 to 40 nm.

In some embodiments, the MaSp-based fiber comprises a plurality of pores. In some embodiments, the porous MaSp-based fiber comprises a plurality of fibrils (e.g. nanofibrils). In some embodiments, the MaSp-based fiber is a form of a particle, as described hereinbelow. In some embodiments, the MaSp-based fiber is as described hereinbelow. In some embodiments, the composition comprises a plurality of MaSp-based fibers. In some embodiments, the plurality of MaSp-based fibers comprises fibers having a different chemical composition and/or a different molecular weight (MW).

As further exemplified in the Examples section below, in some embodiments, a plurality of the disclosed fibers may be in the form of self-assembled structure or matrix. In some embodiments, this matrix can be rendered suitable for biomaterial applications.

In some embodiments, this matrix is suitable for cell growth, and for maintaining or promoting cellular activity, as further demonstrated hereinbelow.

In some embodiments, the term “self-assembled” refers to a resulted structure of a self-assembly process (e.g., spontaneous self-assembly process) based on a series of associative chemical reactions between at least two domains of the fiber(s), which occurs when the associating groups on one domain are in sufficient proximity and are oriented so as to allow constructive association with another domain. In other words, an associative interaction means an encounter that results in the attachment of the domains of a fiber or fibers to one another. In some embodiments, attached domains are not parallel to each other. Also contemplated are arrangements in which there are more than two domains of the self-assembled structure, each engaging a different plane.

It is noteworthy, that in some embodiments, the density of the self-assembled fiber (e.g., about 80% voids) is in the range of from 0.1 g/cm³ to 0.4 g/cm³or from 0.2 g/cm3 to 0.3 g/cm³. In exemplary embodiments, the density of the self-assembled fiber is about 0.26 g/cm³.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Experimental Procedures

### Bacterial Growth

pET24R-expressing bacteria (generated according to procedure disclosed in PCT/IL2020/050752) were seeded into 3 mL starter of LB medium with chloramphenicol and kanamycin, grew until the OD˜0.6 (measured in 100 μL in the 96-well plate), and seeded into a growth medium.

The bacteria was grown at 37° C. under shaking, the OD₆₀₀ and beta sheet specific staining was performed after ˜20 h. Significant beta sheet staining appeared after 24 h. Additional details are disclosed in PCT/IL2020/050752, which is incorporated herein.

Generic Protocol for Enrichment of Polymer with Spider Silk Fiber

A spider silk fiber suspension was centrifuged and re-suspended in a sufficient amount of solvent. The suspension was poured into the polymer solution. The polymer and spider silk polymer suspension was mixed thoroughly until homogeneity was achieved. The solution was then dried.

### Example 1

### Purification SVX Fibers Produced by Bacterial Cells

The bacteria (e.g., pET24R-expressing bacteria) were centrifuged and re-suspended in deionized water. Then, a solution of a surfactant was added, and the resulting suspension was shaken overnight at 37° C. After centrifugation, the pellet (comprising inter alia the isolated MaSp-based fiber) was re-suspended in 6 M Urea. After the centrifugation, the pellet was re-suspended and washed several times with a surfactant solution.

The obtained pellet (isolated MaSp-based fiber) was then washed with t-butanol and the washing was repeated 2-3 times. t-Butanol was then added to the MaSp-based fiber until a homogeneous suspension was obtained. The suspension was stirred to prevent formation of aggregates. After centrifugation, the MaSp-based fiber was dried by lyophilization comprising freezing of the suspension (e.g. in a liquid nitrogen), and applying vacuum to the frozen suspension until complete evaporation of the solvent. The inventors further successfully implemented a mixture of t-butanol and up to 20% w/w water for obtaining a suspension, which upon lyophilization resulted in a lyophilized MaSp-based fiber as described herein. The exact lyophilization conditions may vary, however a skilled artisan will be able to adjust the lyophilization conditions (e.g. the vacuum, lyophilization time, freezing temperature, etc.) so as to obtain the lyophilized MaSp-based fiber, as disclosed herein.

FIGS. 1A, 1C and 1E present the results of fibers obtained after purification and lyophilization with t-butanol, compared to analogous fibers lyophilized from water.

### Example 2

### Mechanical Properties

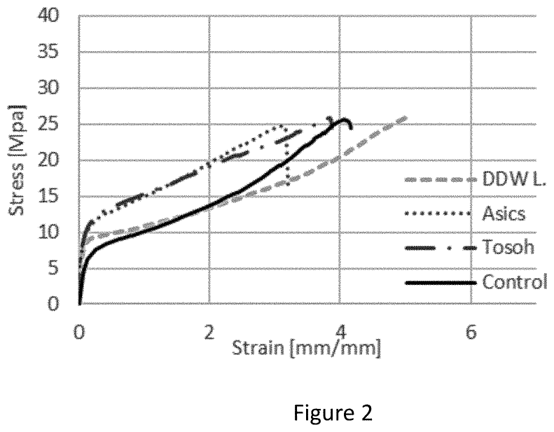

In exemplary experiments, polyuerthane (PU) (E394POTA) was enriched with 15% SVX-E lyophilized from t-butanol or water. The stress-strain curves of PU enriched with 15% SVX-E lyophilized from t-butanol and from water, compared to control (non-enriched PU) are presented in FIG. 2.

### Example 3

### Loading and Release of Hyaluronic Acid

To evaluate the release of hyaluronic acid (HA) from SVX-E lyophilized with t-butanol and lyophilized with water, formulations were prepared with the different SVX-E and HA. Hyaluronic acid (1:1 w/w) was added to SVX-E lyophilized with t-butanol and to SVX-E lyophilized with water.

The release of HA was evaluated by using water washes and the ratio of HA in the complex SVX-HA was measured using Fourier-transform infrared spectroscopy (FTIR).

Graphs, showing a release profile of HA from SVX-E lyophilized with t-butanol and SVX-E lyophilized with water are presented in FIGS. 3A-B.

It can be observed that the SVX-E lyophilized with t-butanol and enriched with HA exhibits a slower release rate of the HA, compared to SVX-E lyophilized with water and enriched with HA.

### Example 4

### Loading and Release of Glycolic Acid

To evaluate the release of glycolic acid (GA) from SVX-E lyophilized with t-butanol and lyophilized with water, formulations were prepared with the different SVX-E and HA. GA (1:1 w/w) was added to SVX-E lyophilized with t-butanol and to SVX-E lyophilized with water.

The release of GA was evaluated by using water washes and the ratio of GA in the complex SVX-GA was measured using FTIR.

Graphs, showing a release profile of GA from SVX-E lyophilized with t-butanol and SVX-E lyophilized with water are presented in FIGS. 4A-B.

It can be observed that the SVX-E lyophilized with t-butanol and enriched with GA exhibits a slower release rate of the GA, compared to SVX-E lyophilized with water and enriched with GA.

### Example 5

### BET Surface Area

Surface areas were determined from nitrogen adsorption data using the BET (Brunauer, Emmett, Teller) method as described in S. Brunauer, P.H. Emmett, E. Teller, J. Am. Chem. Soc. 1938, 60, 309-331. The BET surface area measurements were conducted using, Nova Station A and Quantachrome NovaWin, Quantachrome Instruments version 11.02.

### Sample Preparation

Powder SVX sample of 0.0168 g in 1M1 was heated for 3 hours to 120° C. for outgasing. Analysis was conducted with Nitrogen at bath Temperature of 273° K for 308 minutes.

Surface area of samples of SVX dried from t-butanol and SXV dried from water were measured. The calculated surface area of SVX dried from t-butanol was about 180 m²/g. The calculated surface area of SVX dried from water was considerably lower compared to the surface area of SVX dried from t-butanol, with a calculated value of about 85 m²/g.

### Example 6

### Thermal Stability of Major Ampullate Spidroin Protein (MaSp)-based Polymer

By analysis of the differential scanning calorimetry (DSC) curves of the presented spider silk polymers expressed in bacteria (SVX-E), it can be observed that the SVX-E does not present a melting peak. Instead, it showed small glass transition temperature (T_{g}) regions at approximately 220° C. and at approximately 280° C. It could also be observed a degradation peak at approximately 330° C. (FIG. 5).

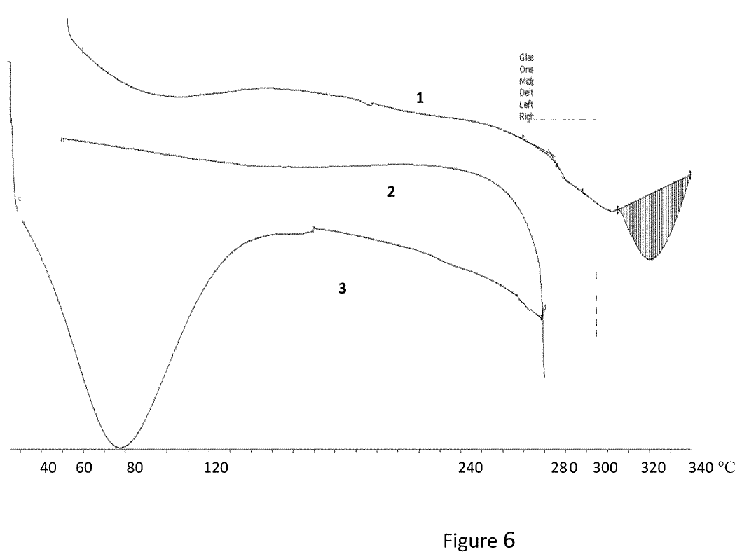

FIG. 6 presents DSC curves of the SVX-E at a temperature increase of 25° C.-280° C. (curve 1), cooling back to 50° C. (curve 2), and new increase to 350° C. (curve 3).

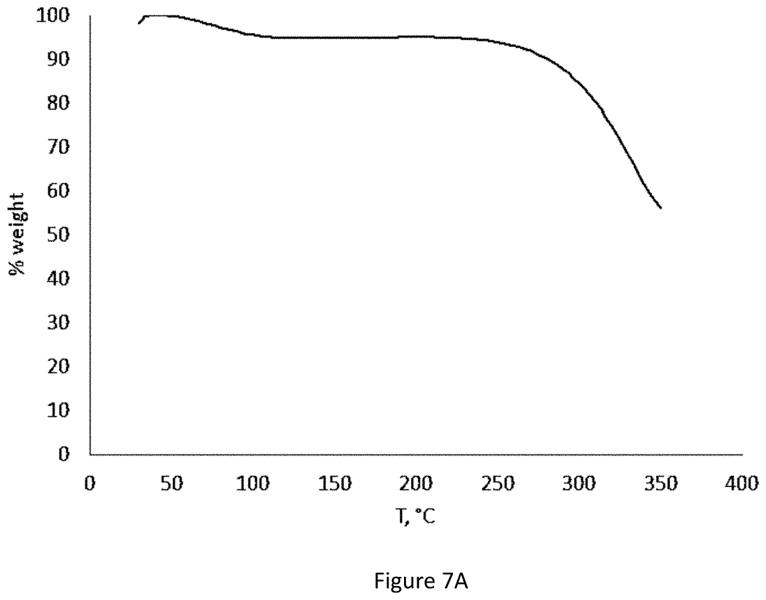

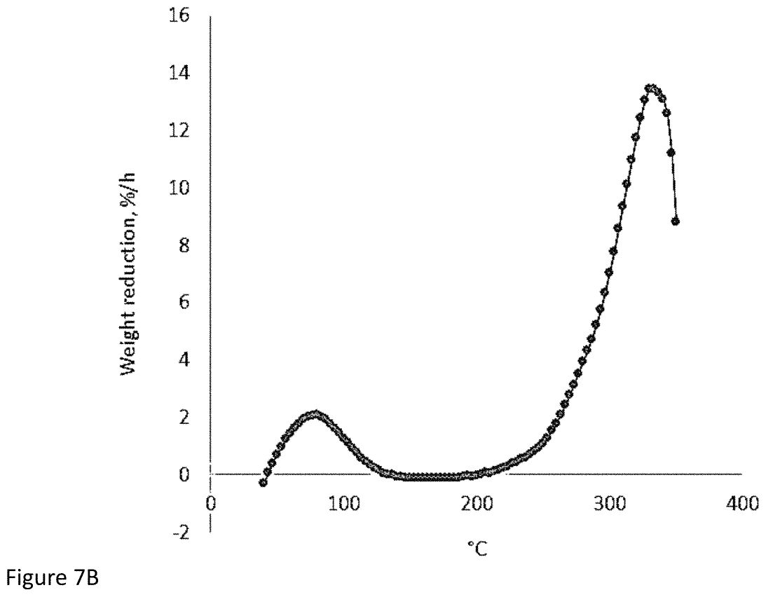

The thermogravimetric analysis (TGA) curves of SVX-E (FIGS. 7A-B) show that at a heating rate of 10° C./min, the weight loss at a temperature bellow 100° C., was only approximately 5% of the absorbed water. It can be observed that a great weight reduction (more than 1%/h) begins at more than 230° C.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising a porous major ampullate spidroin protein (MaSp)-based fiber wherein said fiber is characterized by a BET surface area of at least 100 m²/g, and a residual amount of an organic solvent.

2. The composition of claim 1, wherein said MaSp-based fiber is in the form of particles having a size in the range of 0.5 μm to 2μm.

3. The composition of claim 1, wherein said organic solvent is capable of forming an azeotrope with water.

4. The composition of claim 1, wherein said organic solvent comprises t-butanol.

5. The composition of claim 1, further comprising an additional agent.

6. The composition of claim 5, wherein a w/w concentration of said additional agent within said composition is between 1 and 80%.

7. The composition of claim 5, wherein a w/w ratio between said additional agent and said porous MaSp based fiber is from 100:1 to 1:10.

8. The composition of claim 1, wherein a release rate of said additional agent from said composition is reduced by at least 10%, compared to a control.

9. The composition of claim 1, further comprising a thermoplastic polymer selected from polyester, a polyamide, a polyol, a polyurethane, polyethylene, Nylon, polyolefine, a polyacrylate, a polycarbonate, polylactic acid (PLA) or a copolymer thereof, polycaprolactone (PCL), rubber, cellulose, or any combination thereof.

10. The composition of claim 9, wherein a weight per weight (w/w) ratio of said MaSp-based fiber to said polymer is between 0.01:1 and 1:1.

11. The composition of claim 1, comprising between 0.01% to 50% (w/w) of said MaSp-based fiber.

12. The composition of claim 1, wherein a tensile strength of said composition is enhanced by at least 20% compared to a control.

13. A method for obtaining a dried major ampullate spidroin protein (MaSp)-based fiber, the method comprising:
a. mixing a MaSp-based fiber with a liquid comprising an organic solvent to obtain a mixture; and
b. providing said mixture under conditions suitable for substantially removing said liquid from said mixture,
thereby obtaining said dried MaSp-based fiber, wherein the dried MaSp-based fiber is characterized by BET surface area of at least 100 m²/g, and a residual amount of an organic solvent.

14. The method of claim 13, wherein said liquid optionally comprises an aqueous solvent.

15. The method of claim 13, wherein said organic solvent is capable of forming azeotrope with water, optionally wherein said organic solvent comprises t-butanol.

16. The method of claim 13, wherein said dried major ampullate spidroin protein (MaSp)-based fiber is characterized by a residual amount of an organic solvent, and wherein said MaSp-based fiber is in the form of particles having a size in the range of 0.5 μm to 2μm.

17. The method of claim 13, wherein the dried MaSp-based fiber is characterized by at least one of: (i) an improved loading capacity of an additional agent (ii) sustained release profile, optionally wherein the improved loading capacity comprises a w/w ratio between said additional agent and said porous MaSp based fiber is from 100:1 to 1:10, wherein said additional agent comprises a nutrient.

18. (canceled)

19. (canceled)

20. (canceled)

21. (canceled)

22. (canceled)

23. An article comprising the composition of claim 1, wherein the article is in a form of a reinforced plastic, a container, a packaging material, a cable, a tube, a film, a rope, a thread, or a textile.

24. The article of claim 23, characterized by at least one improved mechanical property as compared to the property for said article free of said composition, wherein said property is selected from the group consisting of: Young's modulus, tensile strength, fracture strain, yield point, toughness, work to failure, impact strength, tear strength, flexural modulus, flexural strain and stress at a specific percentage elongation, abrasion, UV-resistance and gas permeability.

25. The article of claim 23, wherein said composition comprises the dried MaSp-based fiber characterized by at least one of: (i) an improved loading capacity of an additional agent (ii) sustained release profile of said additional agent therefrom, optionally wherein the improved loading capacity comprises a w/w ratio between said additional agent and said porous MaSp based fiber is from 100:1 to 1:10; further optionally wherein said additional agent comprises a nutrient.


SEQUENCE LISTING (8 sequences)

SEQ ID NO: 1 - protein, 555 aa, Artificial Sequence
  Synthetic
AAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGPSGPGASSAAAAAAAASGPGGYGPGSQGPSGPGAYGPGGPGSSAAASGPGGYGPGSQGPSGPGGSGGYGPGSQGPSGPGGPGASAAAAAAAAASGPGGYGPGSQGPSGPGAYGPGGPGSSAAASGPGGYGPGSQGPSGPGAYGPGGPGSSAAAAAAAGSGPGGYGPGNQGPSGPGGYGPGGPGSSAAAAAAASGPGGYGPGSQGPSGPGVYGPGGPGSSAAAAAAAGSGPGGYGPGNQGPSGPGGYGPGGSGSSAAAAAAAASGPGGYGPGSQGPSGPGGSGGYGPGSQGPSGPGASSAAAAAAAASGPGGYGPGSQGPSGPGAYGPGGPGSSAAASGPGGYGPGSQGPSGPGAYGPGGPGSSAAAAAAASGPGGYGPGSQGPSGPGGSRGYGPGSQGPGGPGASAAAAAAAAASGPGGYGPGSQGPSGPGYQGPSGPGAYGPSPSASAS

SEQ ID NO: 2 - protein, 27 aa, Artificial Sequence
  Synthetic
SGPGGYGPGSQGPSGPGGYGPGGPGSS

SEQ ID NO: 3 - protein, 35 aa, Artificial Sequence
  Synthetic
AAAAAAAASGPGGYGPGSQGPSGPGGYGPGGPGSS

SEQ ID NO: 4 - protein, 40 aa, Artificial Sequence
  Synthetic
MSYYHHHHHHDYDIPTTENLYFQGAMDPEFKGLRRRAQLV

SEQ ID NO: 5 - protein, 50 aa, Artificial Sequence
  Synthetic
MSYYHHHHHHDYDIPTTENLYFQGAMDPEFKGLRRRAQLVRPLSNLDNAP

SEQ ID NO: 6 - protein, 54 aa, Artificial Sequence
  Synthetic
MSYYHHHHHHDYDIPTTENLYFQGAMDPEFKGLRRRAQLVDPPGCRNSARAGSS

SEQ ID NO: 7 - protein, 97 aa, Artificial Sequence
  Synthetic
VAASRLSSPAASSRVSSAVSSLVSSGPTNGAAVSGALNSLVSQISASNPGLSGCDALVQALLELVSALVAILSSASIGQVNVSSVSQSTQMISQALS

SEQ ID NO: 8 - protein, 114 aa, Artificial Sequence
  Synthetic
GPSGPGAYGPSPSASASVAASRLSSPAASSRVSSAVSSLVSSGPTNGAAVSGALNSLVSQISASNPGLSGCDALVQALLELVSALVAILSSASIGQVNVSSVSQSTQMISQALS